(19) United States
(12) Patent Application Publication
(10) Pub. No.: US 2008/0058706 A1
(43) Pub. Date: Mar. 6, 2008

(54) Modular electroporation device with disposable electrode and drug delivery components

(75) Inventors: Lei Zhang, San Diego, CA (US); Andre S. Gamelin, Vista, CA (US); Dietmar Rabussay, Solana Beach, CA (US)

Correspondence Address: BIOTECHNOLOGY LAW GROUP;C/O PORTFOLIOIP, PO BOX 52050, MINNEAPOLIS, MN, 55402, US

(73) Assignee: Genetronics, Inc., San Diego, CA (US)

(21) Appl. No.: 11/784,892

(22) Filed: Apr. 10, 2007

Related U.S. Application Data
(63) Continuation-in-part of application No. 11/173,176, filed on Jun. 30, 2005.
(60) Provisional application No. 60/584,816, filed on Jun. 30, 2004.
(60) Provisional application No. 60/588,014, filed on Jul. 13, 2004.
(60) Provisional application No. 60/601,925, filed on Aug. 16, 2004.

(51) Int. Cl. A61N 1/30

(52) U.S. Cl. 604/21

(57) ABSTRACT

The invention comprises a modular electroporation device for use in clinical settings. The device includes components which may be varied or adapted for application of electroporation-based delivery of therapeutic agents to cells of a subject in a variety of electroporation formats such as intratissue electroporation or transsurface electroporation. The device components include a hand-manipulable handle with activation switch and a disposable head comprising electrodes, injection port, electrode directional and depth guide, and a slideably engaged electrode safety shield.

### RELATED APPLICATIONS

Under 37 C.F.R. § 1.53(b), this application is a continuation-in-part of U.S. application Ser. No. 11/173,176 filed 30 Jun. 2005; and under 35 U.S.C. §119(e)(1), this application claims the benefit of Provisional Applications: 60/588,014 filed 13 Jul. 2004; 60/601,925 filed 16 Aug. 2004; and 60/584,816 filed 30 Jun. 2004.

### FIELD OF THE INVENTION

This invention relates to the electroporation arts and particularly devices useful for applying electroporation-based delivery of therapeutic agents to patient tissues and cells. More specifically, this invention relates to electroporation devices capable of delivering therapeutic levels of drugs and other medicaments for treating diseases or application in gene therapy wherein the device comprises modular components, some of which are disposable.

### BACKGROUND OF THE INVENTION

Electroporation has proven to be useful in the delivery of substances directly into biologic cells of tissues. The methodologies employed for electroporation of such materials into tissues have varied and the devices designed for such electroporation have been numerous. However, there remains a need in the art for a clinically-friendly and user-friendly device that can be employed to administer therapeutic agents to patients in need thereof. To date there is no single device designed to have modular components, some of which are intended to be disposable after a single use, that is inexpensive to produce yet highly effective in the clinic and that incorporates various components including a disposable component for carrying a fluid therapeutic or a disposable needle tipped head with safety shield and other functional features. Of particular need is a device that can be easily employed to administer therapeutic compounds to large numbers of patients in a short period of time and while maintaining accuracy in the administration of a therapeutic agent into patient tissues relative to positioning of electrodes in such tissues.

Given the need for a simple, modular and disposable electroporation device, we provide the following invention which will be understood by those skilled in the art to address the ongoing needs in the medical arts.

### SUMMARY OF THE INVENTION

In a first embodiment, we provide an electroporation device for use in administering therapeutic compounds to patient populations in need thereof. In this embodiment, the device comprises a plurality of modular components including a handle, which may be held and manipulated by the hand of the user. The handle comprises a central component of the invention to which is attached at one end an electric wire for electrically connecting the handle to a pulse generator, and at the other end a connector for connecting to the handle a disposable “head” component which also comprises multiple elements.

In preferred embodiments, the elements comprising the head component include any or all of 1) an array of electrodes attached in electrical communication with an electrical connector adapted to mate with the connector of the handle, 2) an injection port and/or injection hypodermic type needle for delivering therapeutic agent into the tissues of a patient, 3) a slidably engaged electrode shield, and 4) an electrode directional and depth guide for aiding predetermined orientation of the electrodes upon entrance into a biologic tissue, and for limiting the depth to which the electrodes and/or injection needle may enter said tissue.

With respect to each of the invention components, each comprise any number and combination of possible structures which may be included and that otherwise provide for variable applications for which the device with its primary modular components (handle and head with electrodes, injection port, shield, and/or depth and direction guide) may be used for medical, veterinary, or clinical research purposes.

For example, in one embodiment, the handle may be designed with specific finger grips such as indicated in pistol grip configuration of FIGS. 1A and 1B. In an alternate embodiment the handle may be linear as shown in FIG. 3A. Additionally, the handle, however shaped, can include a receptacle forming an open-faced indention or trough or alternatively, C-shaped clips, in the upper portion thereof having sufficient dimensions to allow insertion therein and the snug gripping of a hypodermic syringe (with or without an attached injection needle) such that the syringe and its needle are oriented lengthwise in parallel with the electrode array of the head component when the head is connected to the handle, an example of which is depicted in FIG. 2.

In another embodiment, the wire providing for electrical communication between the pulse generator and the handle is attached to the handle such that the wire extends from the handle at an anatomically oriented angle, generally of between 0 and 85 degrees, usually of between 20 and 65 degrees, to the surface of the handle thereby providing for the capability to the user to use the device without the wire interfering or influencing undesirably the user's manipulation of the device. This feature is particularly useful where the handle has a linear construction.

In another embodiment, the handle includes an “activation switch” for activating the electrodes with electrical energy from the pulse generator. Such switch may comprise a trigger e.g., in the form of a pistol trigger or the like in association with a pistol grip, or an activation button positioned for easy manipulation by the user, can comprise a foot switch separate from the hand held device.

In still another embodiment, instead of a trough or C clip to hold a syringe on the top of the invention device, the handle, particularly one constructed as a pistol grip, can include an aperture which extends through the upper portion of the handle for accommodating a plunger capable of being slid back and forth through the handle and for engaging a vial containing a therapeutic compound, said vial further including a slidable piston at one end. (See FIGS. 10A-D and 11A-D). As further described below, the device can be constructed so as to accommodate said vial between the head/electrodes and said handle. When the head, vial and handle are connected together, the plunger may be used to expel fluid from the vial and out of the injection port.

With respect to the head component, elements associated therewith can include numerous variations and modifications including such as follows:

1) Electrode Array. In a preferred embodiment, the electrode array comprises a plurality of individually addressable electrodes mounted on a central core support. The electrodes can be of a type that allows for direct penetration of the electrodes into the tissue of a subject, such as elongated electrically conductive sharply pointed rods or needles, or may be of a type that allows for imparting an electric field through the surface of a subject tissue without the electrodes directly penetrating completely through said surface, i.e., transsurface electroporation-mediated delivery. In embodiments where the electrodes are for transsurface electroporation, the electrodes may comprise non-penetrating needles or rods, microneedles, meander type electrodes or needleless injector electrodes and/or combinations thereof.

In a further embodiment of the electrode array, where said electrodes comprise a plurality of needle electrodes, the array comprises at least four electrodes spaced about a center in roughly a circular pattern. Each of such electrodes is capable of penetrating biological tissue, said electrodes having an even number of electrodes such that there are an equal number of electrodes having opposite polarity. In other words, the electrodes of opposing polarity are “paired” in a spaced relation to one another on opposite sides of the array. The at least four electrodes can be solid or tubular, and if tubular, can be used to inject a therapeutic agent into the tissue. Additionally, if tubular, the electrodes can be fenestrated having export openings at spaced intervals along the length of the electrode and/or at the tip of the electrode. Further still, the electrodes can be energized simultaneously or the electrodes can be energized in predetermined groups. For example, opposed pairs of electrodes can be energized and if more than two pairs of electrodes of opposite polarity are present, the different electrodes can be energized or pulsed selectively around the circular styled array such that the electric field generated during such pulsing of each opposed pair of electrodes is caused to change direction with respect to the area between the array of electrodes.

In still further embodiments, where needle electrodes are used, needle electrodes within an array are spaced at predetermined distances from one another, preferably between about 0.2 cm and 2.0 cm and in a geometric pattern, particularly, a square, rectangle, hexagon, or octagon and oppositely polarizable electrodes are positioned opposite one another on the geometric array. In further related embodiments needle electrodes can be of any length but are generally between 0.4 cm and 5.0 cm long and are between 0.25 mm and 1.5 mm thick.

2) Injection Port. In preferred embodiments, the injection port element of the disposable head comprises an aperture leading to a bore in the central core support placed centrally therein with respect to the array of electrodes as shown, for example, in FIG. 7A. The centrally located bore provides for the placement there through of a syringe hypodermic needle, which may or may not comprise a fenestrated needle. The syringe attached to said needle may be held in place by any number of methods. When in place, the needle further protrudes through the central bore of the central core support and equidistant to each needle electrode. Alternatively, where the syringe is not intended to have a hypodermic needle, the central bore can terminate at the injection port in an injection needle, which may be fenestrated, for dispensing a therapeutic agent into cell containing tissues of a target patient. In such embodiment, the bore leading towards the handle end of the central core support terminates in fluid communication with a channel leading to a universal connector for a syringe.

In still a further embodiment, around the opening of the injection port on the same side as the electrodes, the invention can include a sealing means for making a seal between the central core at the injection port opening (whether the opening is at the surface of the central core support or at the proximal end of a needle, if a needle is present such as by a needle protruding through the bore or a needle directly attached thereto) and the surface of the tissue being treated. The seal provides the capability of avoiding or lessening the loss from an injection channel produced by the insertion of the hypodermic needle into said tissue of material injected in said tissue (such as a patient tissue). The seal may be of any material capable of acting to stop or hinder leakage of fluid material from the site of the injection needle penetration into the tissue of a subject. For example, a seal may be constructed of a resilient material including, but not limited to rubber, plastic, or adhesive.

3) Safety Shield. In preferred embodiments, the safety shield comprises a cowling surrounding the central core (which comprises the interior of the head), such core forming a support for the electrodes and injection port. Generally, the shield forms a “tube” which covers the electrodes when the head is either not attached or attached to the handle but is not ready for immediate use. The shield can be conveniently constructed of a clear or translucent material so that the electrodes and injection port can be viewed therethrough. Further, the shield is slidably connected to the head component central core. In a further related embodiment, whether the head is attached to the handle or not, the shield is maintained in a closed or “safe” position by a tension means, such as, for example, a coil spring or plastic keeper. When the device is prepared for use, the safety shield may be opened by sliding the shield back, exposing the electrodes and injection port. During use, the shield can be maintained in an open position, if desired, by a locking mechanism such as, for example, a spring loaded clasp. Additionally, the head includes a safety shield “guide” attached to the central core near the handle end of the central core and concentric with, and of greater diameter than, the safety shield. The rear or handle end portion of said safety shield, when retracted, slides underneath the shield guide. When fully retracted the handle end of the shield may abut the base of the shield guide/central core interface, i.e., the handle end of the shield abuts a portion of the interior of the shield guide at its handle end where the guide, central core, and electric connector merge together, and thereby limit the travel of the shield.

4) Electrode Guide. In preferred embodiments, and for instances where needle electrodes are used, the guide forms a plate which may comprise the outer end of the safety shield such that said plate has bores therethrough corresponding to each electrode and injection needle. Preferably, said electrode guide provides the ability of the user to keep the electrodes directed on a linear trajectory as the needles enter the patient tissue. Additionally, the guide can be provided on the shield with a predetermined limit to the travel of the shield such that when the shield is opened or retracted, the extent to which it is allowed to slide back can be limited providing for the needle electrodes to protrude past the end of the guide at predetermined depths. The guide may be integral with the outer end of the safety shield or may be a separate modular unit that can be aligned with the needle electrodes and/or safety shield. In the case where the head uses transsurface electrodes, no guide is employed.

In still further embodiments, the handle side of the head within the central portion of the shield guide can be constructed so as to provide a receptacle or cavity in which to place a vial containing a fluid therapeutic agent. In such embodiment, the handle side of the central core bore opening terminates in a short pointed canula capable of piercing a rubber stopper on said vial. Additionally, the vial may be constructed with a movable piston on the end opposite the rubber stopper which when compressed will cause the fluid therein to be expelled out of the injection port.

Further embodiments include an invention apparatus wherein the capability of injecting a therapeutic agent from a syringe or a compressible vial is carried out either fully- or semi-automatically such as by electronically induced actuator or by squeezing a trigger lever as depicted in FIG. 1A. In such embodiment, the action of squeezing the trigger causes the plunger of either the syringe, or a plunger integral with the handle to force fluids out of the syringe or out of a vial and upon reaching a terminal point to which the trigger is squeezed, the electrode activation switch (located on the hand held device or on a foot switch) is activated and the electrodes are energized for electroporating the cells of the treated tissue.

In still further embodiments, the device can be used to treat numerous medical conditions, particularly medical conditions requiring direct delivery of a therapeutic substance into the interior of cells in a biologic tissue. Indications for use of such device include treatment for cancer, vaccination against disease, such as for example by gene therapy, wound healing, etc. In still further related embodiments, the invention device can be used with an outlook to eliciting a predetermined level of histological change in the tissue, such change including changes related to an immune responses in a patient. In this aspect, the voltage, pulse number and length of pulses applied to an electrode array of any given dimensions is predetermined to result in a predetermined electric field strength and pulse duration and repetition pattern to provide a given level of reactivity in the tissue being electroporated. Such reactivity can provide for an appropriate level of immune activation or gene expression in the tissues electroporated.

In another alternate embodiment, the invention device can comprise a head without a handle portion. In this embodiment, novel aspects of electroporation electrode assembly design are provided. In a first aspect of this alternate embodiment, the invention assembly comprises an electrode substrate for mounting a plurality of elongate electrodes. In a preferred embodiment, the plurality of electrodes are mounted in a “plug” which fits into the substrate. In a related embodiment the electrodes are positioned in the plug in a geometric pattern in spaced relation to one another such that they are patterned generally around a circumference centered on a bore in the substrate.

In a second aspect of this alternate embodiment, the bore extends completely through the central core of the substrate so as to form an open-ended port of sufficient dimensions to allow the passage therethrough of a hypodermic needle. In a related embodiment, the bore's central axis lies in a parallel direction with and central to the linear axis of the plurality of electrodes.

In a third aspect of this alternate embodiment, the substrate with electrodes and bore are placed centrally in a substantially cylindrical housing which, due to its dimensions, serves as a safety shield for the elongate electrodes and hypodermic needle, when present. In a preferred embodiment, the substrate in said central placement within said housing is slideably engaged with said housing such that the substrate and electrodes/injector needle can be reversibly moved from a first position wherein the electrodes/injector needle are enclosed within said housing, to a second position wherein at least a portion of the electrodes/injector needle are exposed from one end of said housing. In related embodiments, the housing comprises a clear or translucent material so that the electrodes/needles can be readily viewed. In a related embodiment the housing has opposing slide guide channels running the length of the housing, one each on opposite sides of the housing. Correspondingly, in another embodiment, the substrate has substrate slide tabs which fit into the slide guide channels. In a particularly preferred embodiment, the slide guides and slide tabs keep the travel of the substrate and electrodes in a single linear orientation so that the substrate cannot rotate as it is slid along the shield housing.

In a fourth aspect of this alternate embodiment, electrically conductive leads connect individually each electrode in the electrode plug and terminate in an electric connector port at a position along the circumference of the substrate and extend to a position external to the outer circumference of said housing for connecting the electrodes to a source of electric power. In a further related embodiment, the electric connector port is capable of connection with an external matching plug and wire due to the shield having a cut-out along its length to allow the substrate to slide a predetermined distance with the port so connected to the external plug.

In a fifth aspect of this alternate embodiment, the assembly comprises a connector for engaging the expulsion port of a hypodermic syringe and hypodermic needle attached thereto. Preferably, the connector abuts the bore opening in the electrode substrate on the side of the substrate opposite the electrodes. In a further embodiment, the connector has a channel comprising an open-ended bore that is in-line with the bore of the substrate so that a hypodermic needle, when connected to a syringe, can be removeably placed through both the connector and substrate bores, and the expulsion port/hypodermic needle butt can be engaged with the connector.

In a sixth aspect of this alternate embodiment, the electrode assembly comprises a locking means such that the ability of the substrate to slidably move from said first position to said second position can be forcibly stopped and kept immobile at either of said first or said second position. In a preferred embodiment, the locking mechanism comprises a means which employees a rotation-based locking means which is rotatably engaged with the housing and in rotatably locking or unlocking engagement with the electrode bearing substrate.

A further embodiment the invention device comprises a planar discoid substrate having first and second sides with a plurality of bores therethrough arranged in spaced relation to one another and predominantly in a geometric pattern. In this embodiment, the discoid substrate provides for assisting in the proper orientation of an injected bolus of a fluid medium in a patient tissue relative to the orientation of separately applied elongate electroporation electrodes in said tissue such as by use of said first and alternative embodiments of the modular invention device.

On the first side of said discoid substrate is applied a semi-permanent adhesive material for maintaining the device securely on a preselected surface of a tissue. On the second side, i.e., the side intended to contact electrodes and syringe needle of one embodiment or another of the electroporation apparatus, or of an electroporation device having elongate electrodes without a centrally oriented syringe with injector needle, the surface of said second side is raised to specified dimensions in the areas surrounding the bores so as to extend the internal length of selected ones of said bores. In a related embodiment, the raised portions are designed to predetermined lengths for use, in a first instance, as stops for providing for a predetermined depth of penetration of either of the injection needle or the electroporation needles.

In an additional embodiment, the raised portions, in a second instance, provide for a direction orientation for the needle being inserted therein, whether injection needle or electroporation needle. In a preferred embodiment, the bores are all aligned in a parallel direction in relation to one another.

In another embodiment, the discs can be manufactured according to a color, shape, or other visual code such that different colored, shaped or otherwise coded discs indicate that the disc is designed for a particular depth penetration using particular electroporation needle and injection needle types and lengths. Alternatively, the particular injection needle diameter and length as well as the needle electrode lengths and diameters in millimeters and gages, respectively, for example, can be printed directly on the invention guide.

In yet a further embodiment, the invention guide has orientation markers for use in connection with an electroporation device such that the markers on the guide assist in the orientation of the electroporation device relative to the electroporation needle bores so that the needles can be readily inserted into the bores with ease.

In a related embodiment, the upper end of the bores, whether the injection needle bore or the electroporation needle electrode bores, are funnel shaped to assist in the insertion of the needles into the bores. In this embodiment, the opening of the bores intended for acceptance of the tip of the injection and electrode needles have a larger diameter than the diameters of the needles.

In still further embodiments, the invention guide can be designed to provide for ensuring that the needle electrodes of an electroporation device can not be energized unless the electroporation device is fully inserted into the invention guide. In this aspect, full insertion, which allows for ensuring that the electrodes are properly placed in relation to the injected bolus, can be determined by at least three of the electrode stops abutting the substrate material in which the electrodes are mounted on the electroporation device. When at least three electrode stops of the invention guide are contacted (for electroporation device having at least three electrodes) with the electrode substrate, a signal is sent through the electroporation device allowing the electrodes to become energized upon demand. One of ordinary skill in the art can determine numerous mechanisms by which such contact can provide the required signal for allowing or disallowing an electrical signal to be imparted to the electrodes. In one example, for instance, an electrically conductive surface can be applied at the tips of the stops. When the conductive surface contacts with the electrode substrate near the base of the electrode, the electrical conductive surface completes a circuit between two electrical contacts on the electrode substrate which are situated to be contacted by the conductive stop tip. Completion of a circuit can be used as an electronic signal for allowing firing of the pulse to the electrodes.

In an alternate embodiment of the discoid substrate, only one stop must be contacted with the electroporation device in order to signal that the electroporation device has been fully inserted. In this aspect, the single contact is made using the stop for the injection needle. In a further related aspect, the stops for the electrodes must either be shorter than the injection needle stop, or the electroporation substrate holding the electrodes has an extension centrally located relative to the electrodes such that the tip of the extension can contact the injection bore stop with its electrically conductive tip when the electrodes are fully inserted into the invention guide. Other mechanical means can also be devised to accomplish the same goal of maintaining a safety mechanism of not firing the electrodes unless they are properly in place in the tissue. For example, in addition to relating full insertion with the ability of energizing the electrodes, full insertion can also be related to the contact of the invention guide to a safety shield which is itself a component of the electroporation device. In this embodiment, the invention guide can be designed to “lock” onto a portion of the safety shield, or alternatively a component of the invention guide can comprise an engagement stop which must properly fit the electroporation device safety shield to allow either or both of the safety shield to become disengaged and a pulse signal to the electroporation electrodes.

Other features will become apparent to the skilled artisan from the following description and claims.

### BRIEF DESCRIPTION OF THE DRAWINGS

The application file contains at least two drawings executed in color. Copies of this patent application publication with color drawings, will be provided by the Office upon request and payment of the necessary fee.

FIG. 1A shows one embodiment of the invention handle component comprising a pistol type handle and a lever trigger for actuating an electric pulse emanating from a pulse generator connected to said handle through electric conducting wire 13. FIG. 1B shows a second alternate embodiment of the handle component comprising a finger trigger 12a for actuating an electric pulse and a carrier trough for a syringe needle 16.

FIG. 2 shows the same handle as depicted in FIG. 1B with an upper keeper for connecting the handle to the body of a hypodermic syringe and a syringe attached to the invention device.

FIG. 3A shows an invention device wherein the handle is linear and the head component is designed identically to that for a pistol styled handle. In this embodiment the electrodes are parallel to the length of the handle. FIG. 3B shows an alternative invention device wherein the head is positioned at a ninety degree angle with respect to the length of a linear handle. FIG. 3C shows a breakaway view between the head and handle connection. FIG. 3D shows yet another alternate embodiment wherein a hypodermic needle can be inserted through aperture 104. FIG. 3E is yet another alternate embodiment wherein the head intended for attachment at ninety degrees has electrical connectors on the side of the head for connecting with the handle in a clip-on fashion. In the 90 degree attached head embodiment, a syringe needle can be inserted into aperture 104.

FIG. 4 shows a breakaway depiction of certain components of the head and handle.

FIG. 5A shows a three dimensional model of one embodiment of the head component wherein said head has needle electrodes covered by the safety shield, a shield guide, a tension spring, and a connector end of the head on the handle side of said head component, said connector comprising the terminal ends of the electrodes which are intended to mate with the connector of the handle. FIG. 5B shows the same three dimensional view but from the handle end of the head and in a cut away fashion so as to expose one example of the positioning of the channel leading from the central core support to the syringe connector. In FIG. 5B, a cut away depiction of the head is shown with the safety shield retracted. Also the electrode connectors on the handle side of the head are displayed along with a syringe attached to the head.

FIG. 6 shows a detailed drawing of one manner in which a syringe can attach to the bore on the handle end of the central core support. In this embodiment, the channel connects the bore to a syringe.

FIG. 7A shows the head component with the safety shield retracted (as indicated by the compressed spring shown in the cut out view) so that the injection port is visible with a seal surrounding the port (the electrodes are not shown for clarity of the seal). FIG. 7B shows a break down of the inner portions of said head component wherein needle electrodes are shown surrounding said seal of said injection port. FIG. 7C shows a head with meander type electrodes surrounding said seal and FIG. 7D shows a head having microneedle (or shallow surface-tissue penetrating) electrodes surrounding said seal.

FIG. 8 shows a three dimensional depiction of a head component with a safety shield, coil spring tensioner and shield guide.

FIG. 9A shows one embodiment of the head component wherein said safety shield has an orientation slot and FIG. 9B shows that the central core support has a tang which fits into said slot for keeping the shield in proper orientation relative to needle electrodes in embodiments wherein the electrode guide is integral with the outer end of the safety shield. FIG. 9C shows one embodiment wherein a needle direction and depth guide is separate from the safety shield.

FIG. 10A shows a three dimensional view of the handle end of the head wherein there is a receptacle 120 within the shield guide for accepting a cylindrical vial. FIG. 10B shows a breakaway drawing of the head, a vial for inserting into receptacle 120 and the handle with a plunger 124. FIG. 10C is a partial cross section of the head showing the inserted vial in the head. FIG. 10D shows a cross sectional drawing of a vial for inserting into the invention head.

FIG. 11A shows one embodiment of the invention including actuator 130 which can be used to cause a syringe plunger or an internal plunger to depress. FIG. 11B shows the trigger compressed which in turn causes the plunger actuator to depress the plunger. FIGS. 11C and 11D show cross sections of one embodiment for the actuator mechanism. Briefly, upon squeezing the trigger, lever 132 rides upon cam follower surface of 133 which when fully actuated, causes the end of the plunger 134 of plunger 124 to press upon the piston of the vial.

FIG. 12 is a bar graph showing that gene expression is similar after intramuscular injection of DNA with needle and syringe followed by electroporation, regardless of whether a 1 cm or 0.5 cm diameter 4 needle array is used in electroporation.

FIG. 13 shows one embodiment of a system comprising an electric pulse generator, and a linear handle device with a disposable head, and connecting wire between the generator and handle of said device.

FIG. 14 shows a pain scale used in Example 1.

FIG. 15 is a bar graph depicting results of a pain study using 1.0 and 0.5 cm diameter electrode arrays.

FIG. 16 is a bar graph depicting the effects of electric pulse cycles and time after treatment on the necrosis found in the subcutaneous muscles following administration of saline or bleomycin and subsequent electroporation. The effects of both the number of pulse cycles (P<0.0001) and days after treatment (P<0.0001) had a significant effect on the severity scores. Histological changes were most noticable on days 1 and 5, and in sections receiving 4 or more pulse cycles. Data represent severity scores (scored on a scale from 0 to 5).

FIG. 17 is a bar graph showing the effects of the needle array diameter and time after treatment on the necrosis found in subcutaneous muscles following administration of saline or bleomycin and subsequent electroporation. The effects of both the needle array diameter and time after treatment had a significant effect on the severity scores. Histological changes were most severe early after treatment and in sections treated with the needle arrays of largest diameters. Data represent severity scores (scored on a scale from 0 to 5).

FIGS. 18A and B are bar graphs showing the effects of the needle array diameter and time after treatment on the necrosis found in subcutaneous muscles following saline (FIG. 18A) or bleomycin (FIG. 18B) injection and subsequent electroporation. Data represent severity scores from 0 to 5.

FIG. 19 is a bar graph showing the effects of electric pulse cycles and time after treatment on the inflammation found in the subcutaneous muscles following administration of saline or bleomycin and subsequent electroporation. The effects of both the number of pulse cycles and time after treatment had a significant effect on the severity scores. Inflammation was mild or minimal, except in sections receiving more than 2 pulse cycles and collected on day 5 after treatment. Data represent severity scores on a scale of 0 to 5.

FIG. 20 is a bar graph showing the effects of needle array diameter and time after treatment on the inflammation found in the subcutaneous muscles following administration of saline or bleomycin and subsequent electroporation. The effects of both the needle array diameter and time after treatment had a significant effect on the severity scores. Histological changes were most severe on day 5 and in sections treated with the needle arrays of largest diameters. Severity was scored on a scale of 0-5.

FIG. 21 is a bar graph showing the effects of electric pulse cycles and time after treatment on subcutaneous muscle hemorrhage following administration of saline or bleomycin and subsequent electroporation. The effects of both the number of pulse cycles and time after treatment had a significant effect on the severity scores. Histological changes were minimal to mild, except on day 5 and in sections receiving more than 2 pulse cycles. Data represent severity scores on a scale of 0-5. Hemorrhage change was minimal to mild and was not found on or after day 40.

FIG. 22 is a bar graph showing the effects of needle array diameter and time after treatment on subcutaneous muscle hemorrhage following administration of saline or bleomycin and subsequent electroporation. The effects of both the needle array diameter and time after treatment had a significant effect on the severity scores. Hemorrhage was mild but consistently found in samples treated with the 1.35 cm needle arrays; with other types of arrays, no hemorrhage was found after day 10. Data represent severity scores on a scale of 0-5. This change was minimal to mild and was not found on or after day 40.

FIG. 23 is a bar graph showing the effects of electric pulse cycles and time after treatment on muscle fibrosis found following administration of saline or bleomycin and subsequent electroporation. The time after treatment has a significant effect on the severity scores. The number of pulses greater than 1 had no significant effect, except on day 5 when changes were most severe. Severity was scored on a scale of 0-5.

FIG. 24 is a bar graph showing the effects of needle array diameter and time after treatment on the fibrosis found in the subcutaneous muscles following administration of saline or bleomycin and subsequent electroporation. The effects of both the needle array diameter and time after treatment had a significant effect on the severity cores. Fibrosis was most severe in samples treated with needle arrays of the largest diameter, and persisted in mild form throughout the course of this study. Severity was scored on a scale of 0-5.

FIG. 25 is a bar graph depicting the effects of electric pulse cycles and time after treatment on the epidermal damage found following administration of saline or bleomycin and subsequent electroporation. The time after treatment had a significant effect on the severity scores. The number of pulse cycles greater than 1 had no significant effect. This change was minimal in most samples and only mild on day 1. Severity was scored on a scale of 0-5.

FIG. 26 is a bar graph showing the effects of needle array diameter and time after treatment on the epidermal damage found following administration of saline or bleomycin and subsequent electroporation. There was no direct effect of needle array diameter. This change was minimal in most samples and mild only on day 1. Severity was scored on a scale of 0-5.

FIG. 27 is a bar graph depicting the effects of electric pulse cycles and time after treatment on the subcutaneous inflammation found following administration of saline or bleomycin and subsequent electroporation. The time after treatment had a significant effect on the severity scores. Changes were most noticeable in samples receiving 2-8 pulse cycles, and changes were most severe on days 1, 10, and 20. Severity was scored on a scale of 0-5.

FIG. 28 is a bar graph showing the effects of needle array diameter and time after treatment on the subcutaneous inflammation found following administration of saline or bleomycin and subsequent electroporation. There was no direct effect of needle array diameter on this change, and it could be found in a very mild form throughout the study. Severity was scored on a scale of 0-5.

FIG. 29 is a graph depicting glycoprotein D-specific antibody responses in pigs following immunization. BHV-1 neutralization titers were determined in serum at 6 weeks. Numbers above the groups indicate the number of animals for which clinical signs of infection would be expected to be reduced (greater or equal to 32 BHV-1 neutralization titer). Groups 3-5 were significantly different vs. prebleed, P<0.01 by chi-square test, whereas Groups 1 and 2 were not significantly different compared to prebleed.

FIG. 30 is a graph depicting cellular immune responses in gD-immunized pigs assessed by proliferation and IFN gamma cytokine secreting cells. Glycoprotein D-specific proliferative responses were determined at week 6. The number of gD-specific IFN-gamma secreting lymphocytes was determined using ELISPOT at week 6. Error bars represent standard deviation (SD) and n=6.

FIG. 31 is a graph depicting the effect of electoporation on anti-HBsAg titers in pigs following immunization. Anti-HBsAg titers were determined using an ELISA test. Numbers above the groups are the number of animals considered protected (greater than or equal to 10 mIU/ml). Groups 2 and 4 vs. prebleed P<0.01 by chi-square test, whereas Groups 1, 3 and 5 were not significantly different compared to prebleed.

FIG. 32 is a cross section drawing of one embodiment of the electrode assembly showing the electrode bearing substrate with attached hypodermic syringe and needle in a first position wherein the electrodes are within the safety shield housing.

FIGS. 33A and B are external drawings of the electrode assembly showing in FIG. 33A the electrodes retracted within the shield and in FIG. 33B the electrodes positioned external to the shield. Also shown in the figures is the locking pin guide channel in the substrate and the electric plug port.

FIG. 34 is a drawing of a typical electroporation device comprising a linear handle and array of electroporation needle electrodes in a 90 degree relation to the handle. The electrode needles can be inserted into the multiplicity of bores in the guide disc. This embodiment does not include a safety shield.

FIG. 35 shows an alternate embodiment wherein an electroporation device with a linear handle and electrodes positioned parallel with the handle is used with the guide disc.

FIG. 36 is a drawing showing that the side of the disc intended to be place against a surface tissue is planar and has a layer of an adhesive applied thereto such as that type commonly used on tape or band aides.

FIG. 37A shows a three dimensional view of the intended placement of a hypodermic injection needle, the bolus of fluid injected therefrom and the placement (dotted lines) of electroporation needles. FIG. 37B is a cross section drawing showing the placement of a fully inserted hypodermic needle and the relation of the injected bolus to the placement of the electrodes.

FIG. 38 shows a cross section drawing after the syringe is removed and the needle electrodes are inserted, in this example the syringe depth guide serves as a stop for the depth of the electroporation needles.

FIGS. 39A-C show alternate embodiments of the guide disc. FIG. 39A shows a disc wherein the guide stops for the electrodes are raised to a dimension longer than the injection needle stop. In FIG. 39B, the electrode guide stops are substantially longer than the injection needle stop. In this instance the upper extension of the electrode guides can include a planar support band forming such as a donut shape that will allow a syringe to be inserted therein. In FIG. 39C, an embodiment is displayed wherein all of the electrode and injection needle guides are of equal length.

FIG. 40 shows one embodiment of the invention guide wherein the top portion of the substrate for the electrode bores has applied thereto an electrically conducting surface.

FIG. 41A shows a line up of the guide with electrically conductive surface on the top portions of the electrode guides and the placement of electrical conductors around the base of selected electrodes on the electroporation device. FIG. 41B shows the electroporation device fully inserted such that electrical contacts meet one another.

FIG. 42A shows another embodiment wherein the invention guide can, in a properly orientated fit with an electroporation device comprising a safety shield, by such proper fit allow for the safety shield to retract prior to energizing the electrodes as shown in FIG. 42B.

### DETAILED DESCRIPTION OF THE INVENTION

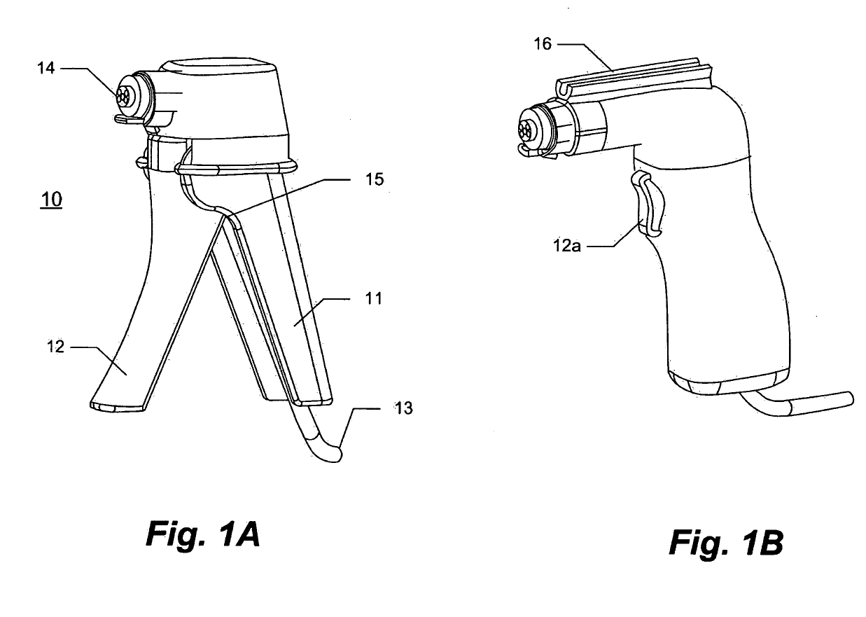

In a first embodiment, the invention can comprise a handle component wherein said handle has various embodiments. For example, in one embodiment, as provided in FIG. 1A, handle 10 comprises a pistol grip 11 and a trigger lever 12. Attached to either the base of the grip 11 or at the rear of the handle above the grip is connected electrical lead 13. The handle further comprises electrical connector 14 for attaching a disposable head component. Additionally, in a further embodiment, the handle portion includes an activation switch 15 (hidden under upper portion of said trigger 12) which is in electrical communication with the pulse generator. Alternatively, the handle portion may include a pistol grip and pistol type trigger 12a as shown in FIG. 1B. When the switch is closed, electric energy is imparted to the electric connectors on the handle for connecting the head component. In a preferred embodiment, the switch is activated by a trigger or a button type switch.

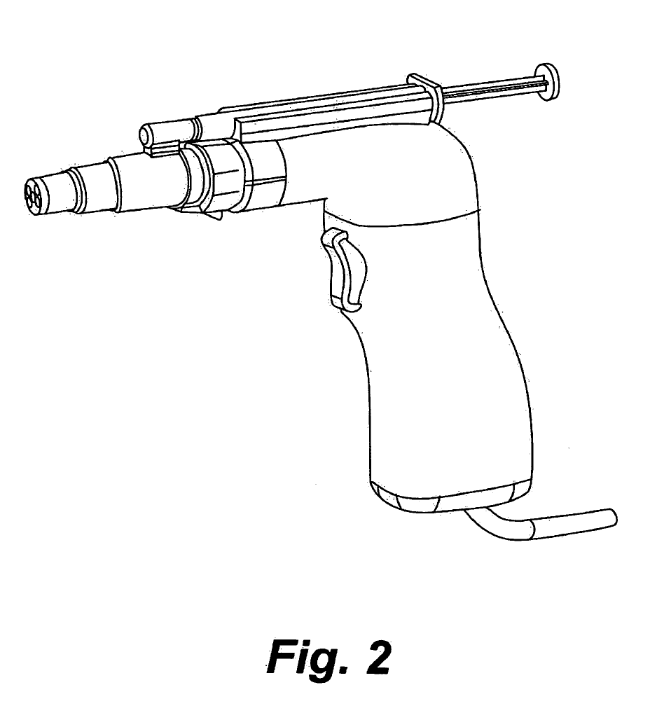

In another embodiment the handle component includes an upper receptacle in the form of an open-ended trough 16 of sufficient dimensions to accommodate the main body of a typical hypodermic syringe as shown in FIG. 1B and FIG. 2. In this embodiment, the handle is intended to be used with a disposable head comprising electrodes, which can be of any type including needle, meander, microneedle, or needleless injector. In use, the body of the hypodermic needle is placed into the receptacle or trough while the needle end of the syringe is attached to a connector on the head component, the combination of trough and connector allowing for removable attachment of the syringe. The syringe body is maintained in the trough using any method including, but not limited to friction, clamp, clasp. (See FIG. 2) When this format of the invention is applied, the device user can 1) place the electrodes on (in the case of meander electrodes) or in the patient tissue (in the case of elongate needle electrodes) with hypodermic needle inserted, 2) inject the substance in the syringe using a finger or thumb, and 3) squeeze the trigger to initiate the electric pulse for electroporation, followed by 4) removal of the device from the patient tissue.

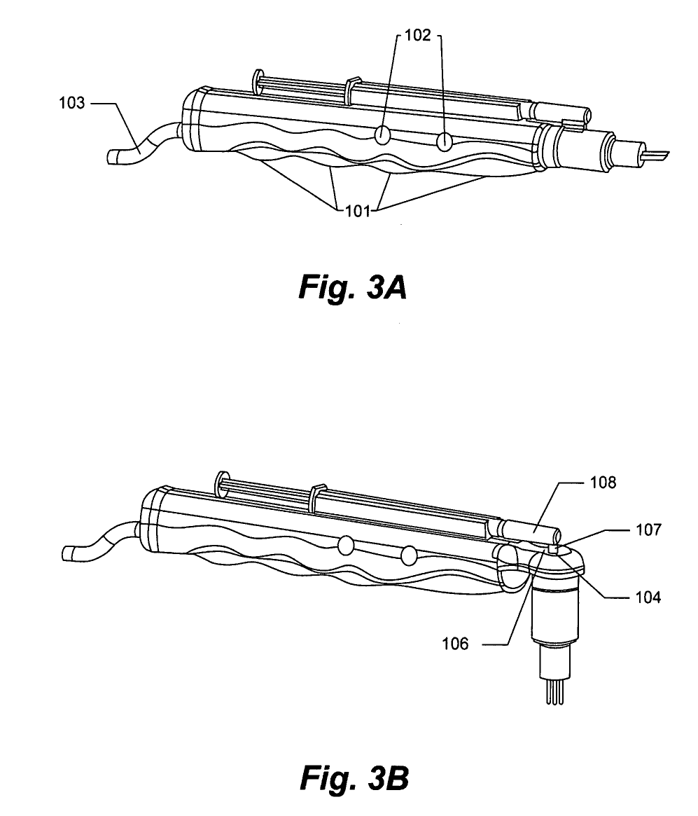

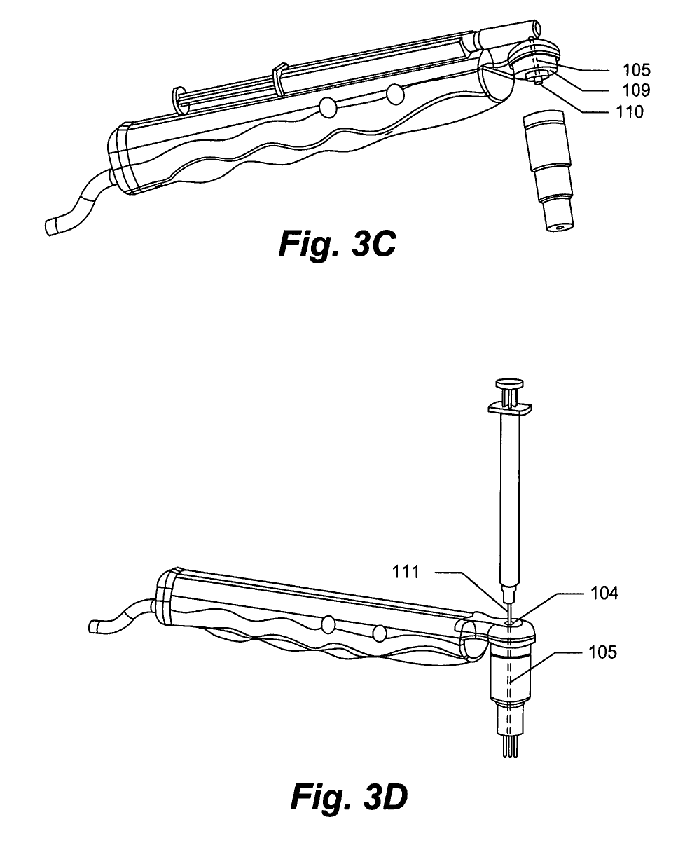

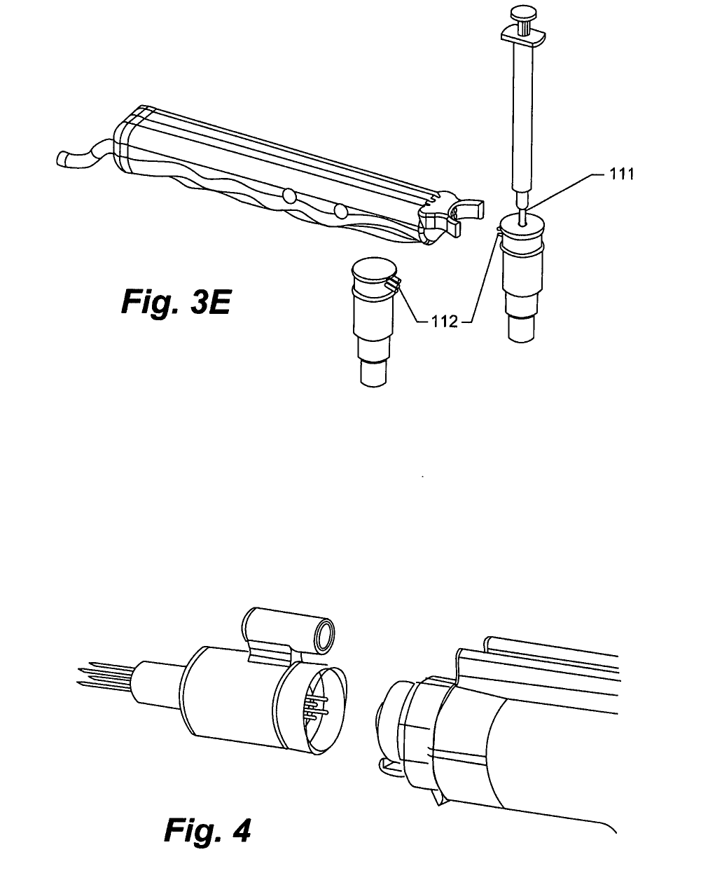

In one embodiment, when the head is connected to the handle, a syringe carrying its own needle of appropriate dimensions is brought into proximity with the invention and in an alternative embodiment instead of a syringe connector as in FIG. 2, the syringe, including its own needle, is inserted into a bore opening on the handle side of the central support of the head component and slid through the bore such that when fully inserted, the needle exits the injection port to a predetermined distance out of the port. In such application, the handle preferably comprises a linear type handle with the head situated at a ninety degree angle with relation to the handle. See FIGS. 3B-E.

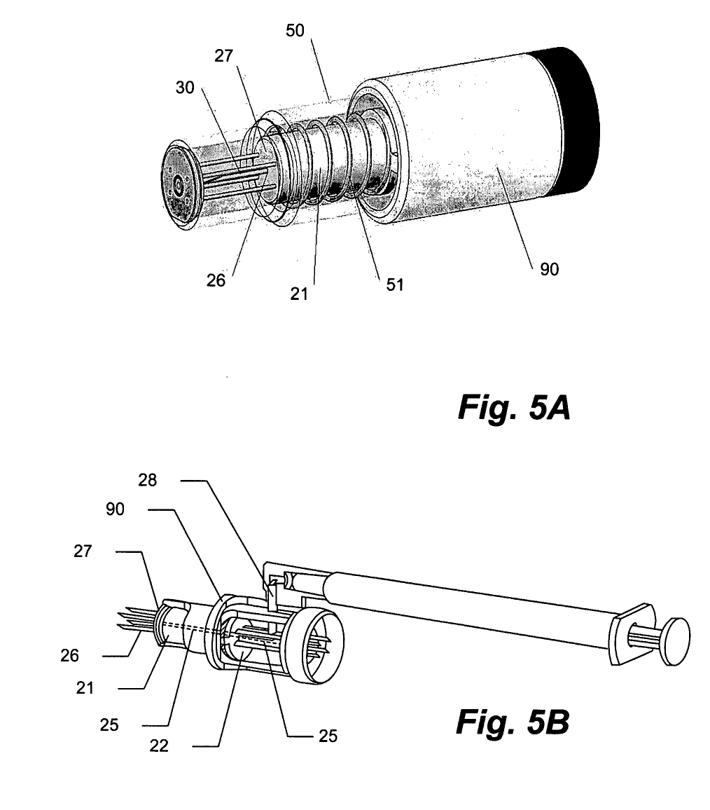

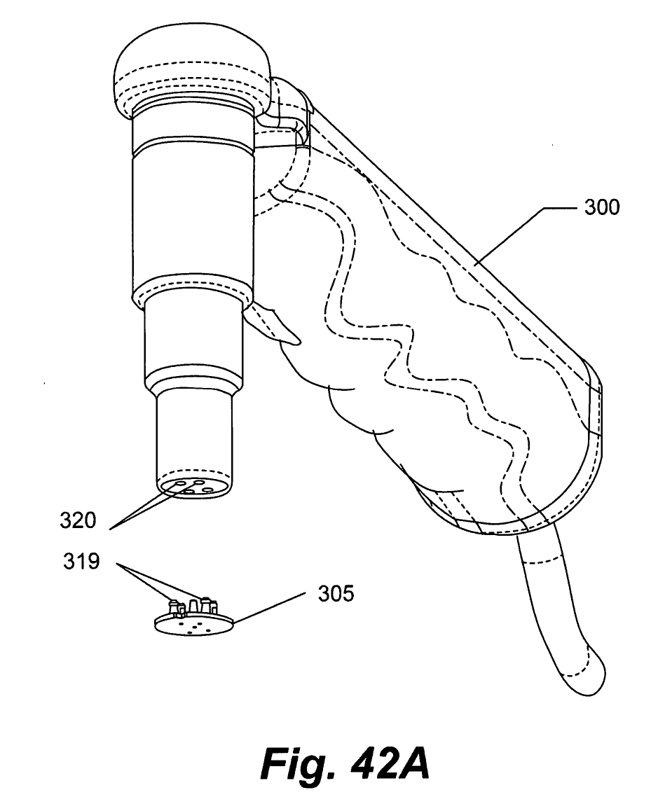

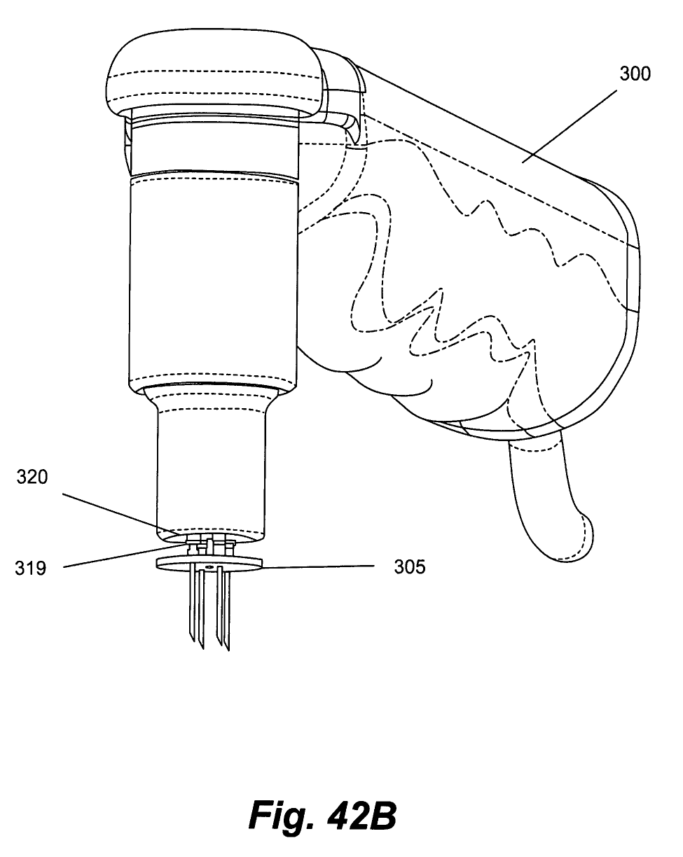

In still another embodiment, as shown in FIG. 3A, the handle 100 can be linear with appropriate modeling for finger grips 101, whether sculptured or rubberized. The handle can also support button switches 102 as needed for initiating a closed circuit between the pulse generator and electrodes. Additionally, the electric lead 103 to the pulse generator can be fitted to the handle with ergonomic considerations factored. For example, the lead 103 can extend away from the end opposite the head at an appropriate angel of between 40 and 60 degrees. Further, the handle can support the body of a syringe 104 and the head component can be attached to the handle as with other head/handle embodiments. In this embodiment, however, the head can either be attached in-line such that the electrodes extend opposite the handle end of the head as in FIG. 3A, or can be directed at a 90 degree angle with the linear lengthwise axis of the handle as depicted in FIGS. 3B-E. In an embodiment wherein the head is attached parallel to the length of the handle, the head component can comprise a shield guide surrounding a safety shield, which surrounds the central core and a connector extending therefrom to a syringe connector, i.e., essentially the same head as used in an embodiment wherein the handle is a pistol grip type handle, (see FIGS. 5, and 42A for example).

In embodiments wherein the head connects to the handle at a 90 degree angle, the handle can include an aperture 104 which comprises a bore 105 that, when the head is attached to said handle, continues by direct connection thereto to the bore 25 of the central core support 21 of the head. The aperture 104 can either connect on a top side 106 of the handle to a closed channel 107 leading to a syringe connector 108 while on a bottom side 109 the bore 105 opening connects to a connector 110 for sealably connecting the end of the bore 25 on the handle side of the central core to the handle, said connector sufficient to seal the contact between the central core and the handle from leakage of fluids there between. Alternatively, the aperture 104 on the handle may, as depicted in FIG. 3D, simply be of sufficient diameter to allow a hypodermic needle 111 to pass therethrough and continue through the bore 25 in the central core support 21 to a predetermined position beyond the end of the electrode side of the central core support.

In still yet another embodiment, as depicted in FIG. 3E, the heads for a linear handle device may be constructed such that the electric connection 112 between the head and the handle is positioned at the side of the cylindroid safety shield guide as depicted in FIG. 3E. In this embodiment the head central core support bore opening on the end opposite the electrodes could be sized to accommodate a hypodermic syringe needle 111 such as one sized of an appropriate diameter and length predetermined for the intended treatment therewith.

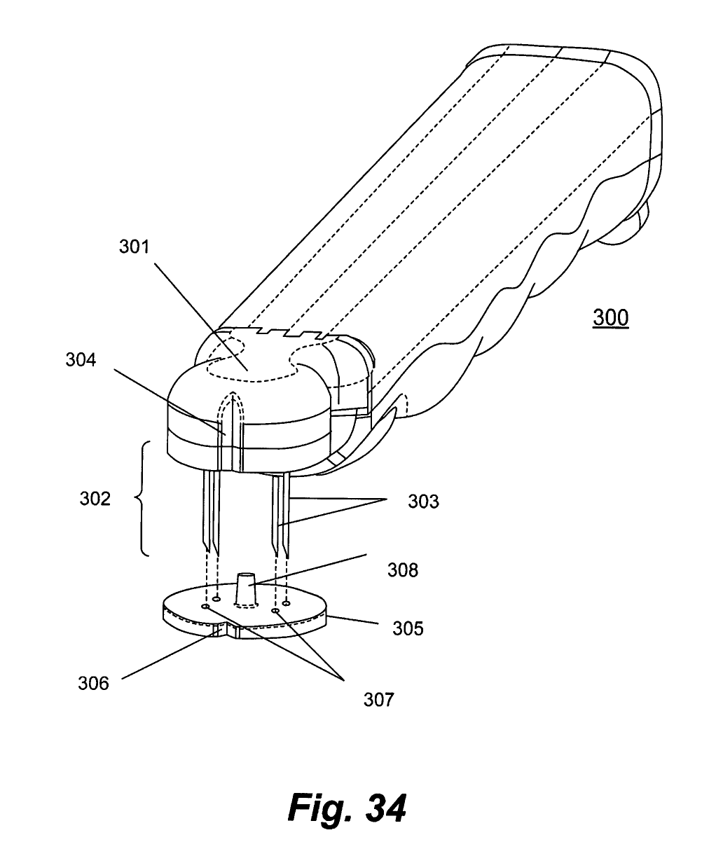

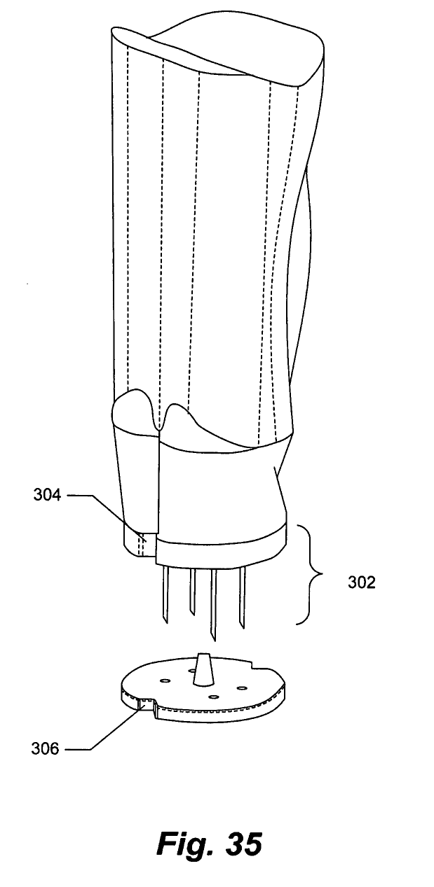

In still other embodiments, the head component need not include a retractable shield or syringe port but instead include a plurality of electrodes which may be guided into tissue using a discoid guide as depicted in FIGS. 34 and 35.

Turning now to the head component, in a first embodiment, as depicted in FIG. 4, the head 20 can comprise multiple modular elements including a central core support 21 having first and second ends and a central body. On the first, or handle, end 22 of the core extends terminals for each of the cathode and anode electrode lead elements 24 which comprise the electrical contacts for connector 23 which is designed to mate with handle connector 14 and provide electric communication between the electrodes and the pulse generator.

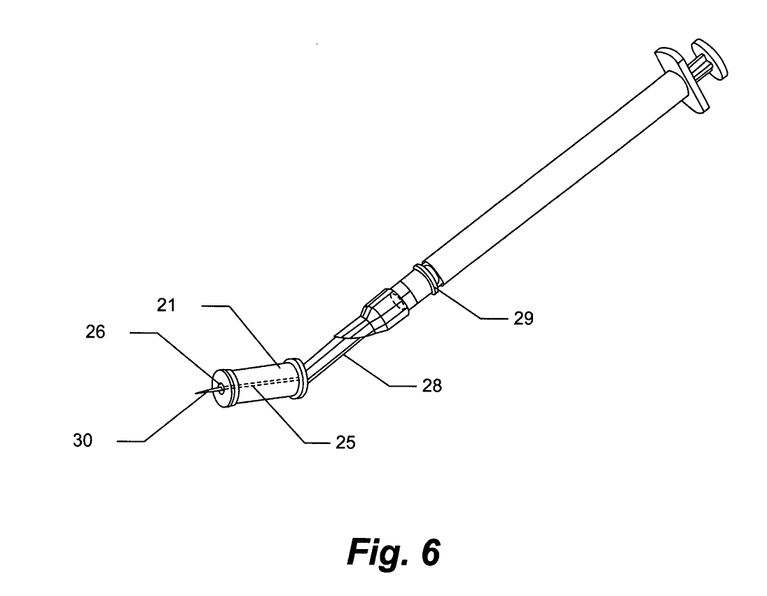

As depicted in FIG. 5A, connected with the central core support 21 and on or near the handle end 22 is the opening of a bore 25 (see FIG. 5B). This bore extends through the central body of the core support and ends at the injection port 26 on the second, or therapeutic, end 27 of the central core support 21. In one embodiment of the invention, the bore 25 is of a diameter sufficient for insertion of a hypodermic needle there through. In an alternate embodiment, as shown in FIG. 5B and FIG. 6 the bore 25 connects to an enclosed channel 28 having first and second ends, said channel capable of carrying in fluid communication a liquid, said channel terminating at said second end in a connector 29 for a hypodermic syringe. The enclosed channel 28 may be constructed of any material useful for carrying a therapeutic solution and can include, but not be limited to, flexible tubing, plastic or metal. In a further related embodiment, where it is desired to inject a therapeutic substance into the tissues of a patient, where a connector 29 is used to connect a syringe it may be desirable to have an injection needle 30 already attached to the injection port 26. As with a needle connected directly to a hypodermic syringe and positioned through the bore as described above, said needle on said syringe, or said needle 30 attached to the injection port may be fenestrated.

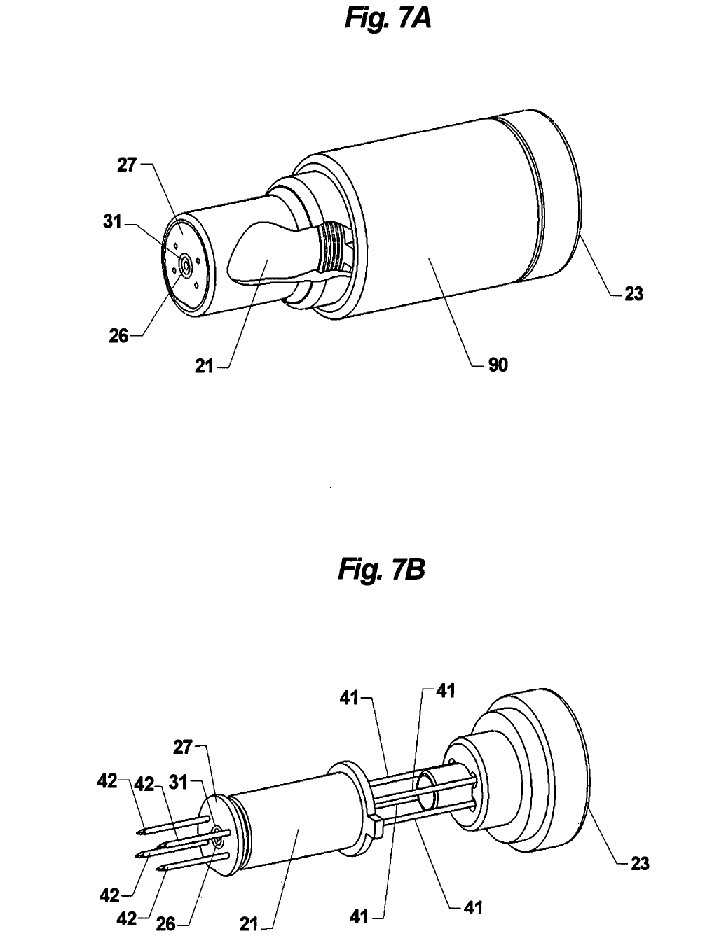

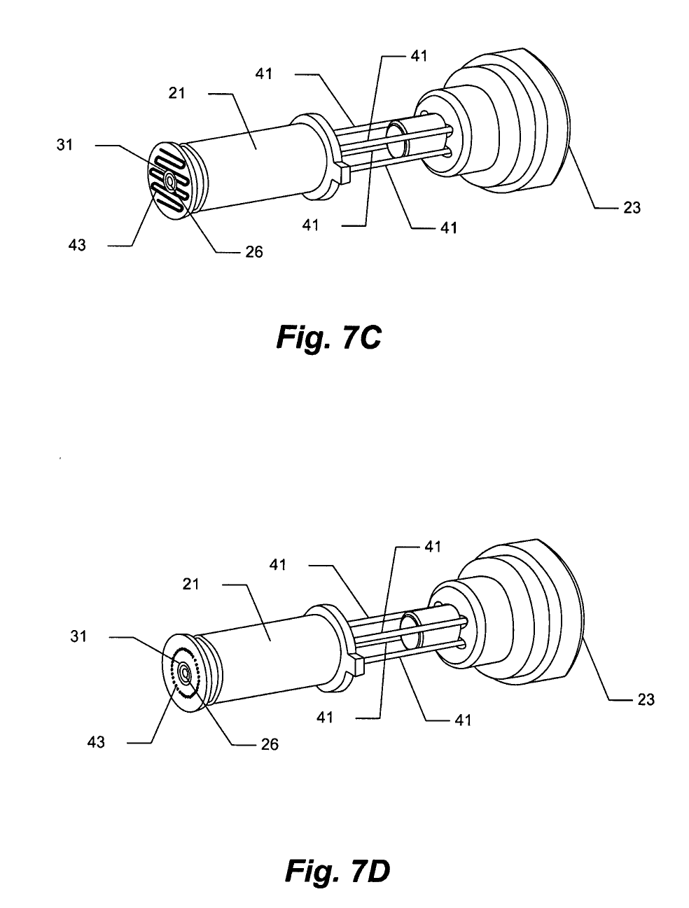

Turning now to FIG. 7A, on the therapeutic end 27 of the central core 21 and surrounding the injection port 26 is a seal 31 made of a resilient material that provides for assisting in keeping fluids ejected out of the injection port 26 and into biologic tissue from leaking out of the tissue following injection.

In a further embodiment, as depicted in FIG. 7B on the therapeutic end 27 of the central core support 21 and surrounding said injection port 26 and seal 31 are a plurality of anode and cathode electrodes. The electrodes can comprise any of needle, meander and microneedle type electrodes, examples of which are depicted in FIGS. 7B-D, respectively. With respect to needle electrodes, the electrodes are connected to the connector 23 via an electric leads 41. Depending upon the application, the head component can be constructed with a variety of electrodes. In one embodiment, as depicted in FIG. 7B the electrodes can accommodate electroporation of internal tissue cells using needle type electrodes 42. Alternatively, as shown in FIG. 7C, the electrodes can comprise non-tissue penetrating type electrodes 43 as are found in the form of meander type electrodes or needleless injector electrodes. In a further alternative embodiment, as depicted in FIG. 7D, the electrodes can comprise semi-penetrating surface electrodes 44 as are found in the form of microneedle electrodes. In each instance, the electrodes are connected to the central core support 21 in electrical communication with connector 23.

In a further embodiment shallow surface semi-penetrating and penetrating electrodes are coated with gold which may be of a thickness between 0.5 and 20 microns. Gold coating provides for avoidance of toxic metallic contamination in the tissue from the act of energizing the electrodes.

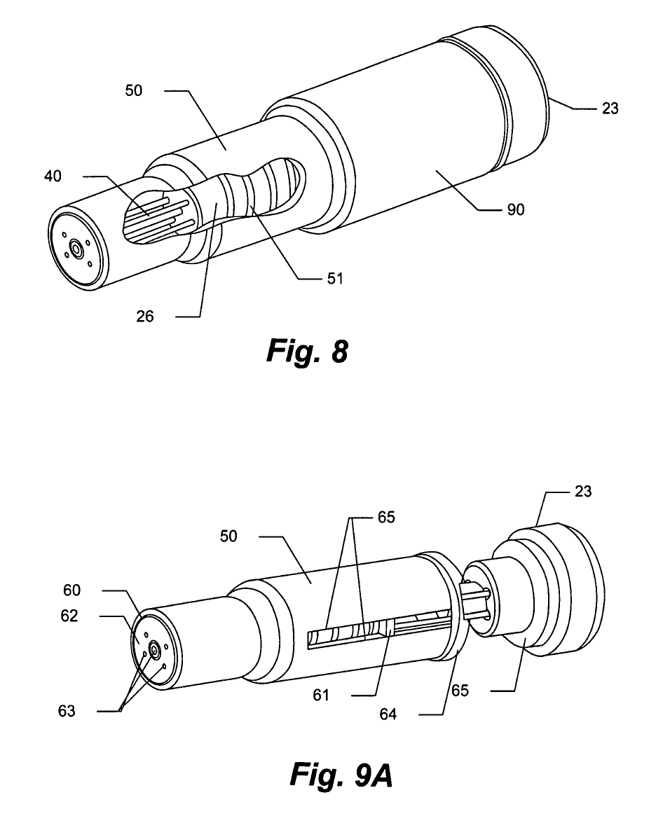

Additionally, as shown in FIG. 8 the invention head component further comprises a safety shield 50 which surrounds the central core support 21 and is capable of traveling in both directions thereover. The safety shield 50 preferably is made of a clear or translucent material so that in the first instance, the electrodes and injection port (needle present or not) can be viewed therein from the outside. This allows for a user to observe the injection port and needle if attached or if extending out of said injection port to check for purging of any air from the port/needle. The end of the safety shield extending on the therapeutic side of the central core support 21 and electrodes 40 provides safety not only from accidental needle stick, but also against accidental contact with the electrodes or sterile injection needle. The safety shield 50 is also maintained in a closed or “safe” position by a coil spring 51 or equivalent tension-based method and may be opened by simply sliding the safety shield, against said tension. Typically, an operator will arm the device by attaching the appropriate head to the handle, then attach the means of deploying a therapeutic agent, such as a syringe (either connected to a syringe connector 29 or sliding a syringe with needle attached through said bore 25 and clamping the syringe to the top of the handle), followed by pressing the device against the patient/surface tissue which in turn causes the safety shield to slide back allowing exposure of the electrodes and injection port from under the shield.

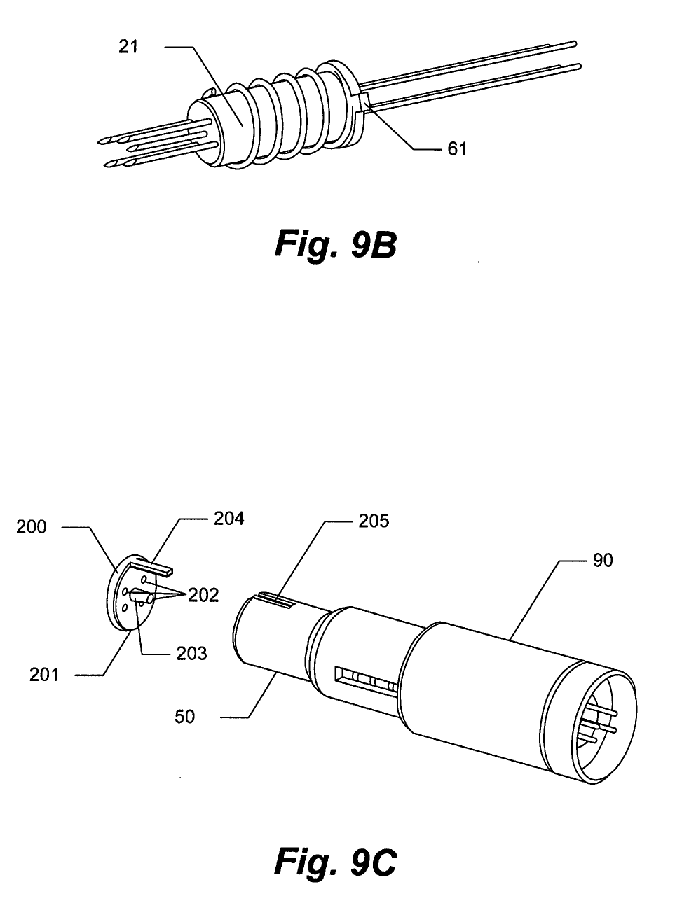

In further embodiments using either needle electrodes and/or injection needle (whether connected directly to the injection port or attached directly to a syringe), the invention safety shield 50 can include on its terminal end a needle electrode direction guide 60 (see FIG. 9A; no safety shield guide surrounding the safety shield is shown) and an orientation slot 66. With respect to the needle direction guide 60, the guide comprises a planar “cap” 62 on the end of the safety shield 50 such that said end cap 62 is in a plane ninety degrees with respect to the needle electrodes and further has a plurality of bores or apertures 63 therethrough to accommodate the placement therethrough of said electrodes and injection needle if present. This direction guide 60 provides support to the electrodes such that when the end cap is placed against the tissue to be treated, the guide keeps the orientation of the needles in a linear and parallel direction in relation to one another as the electrodes enter the tissue. In a related embodiment the direction guide 60 is intended to work in conjunction with the safety shield's 50 orientation slot 66. Specifically, orientation slot 66 comprises a slot 66 into which tang 61 (which is mounted on the central core support 21 as shown in FIG. 9 B) fits. This orientation slot 66 provides for the safety shield to remain in proper orientation such that the apertures 63 in the direction guide 60 remain properly aligned with the electrodes as the safety shield is slidably moved to expose the electrodes.

In an alternate embodiment, the direction guide can comprise a modular component 200 separate from the safety shield but capable of interacting with the safety shield as shown in FIG. 9C and FIGS. 34 through 42. In this aspect, the electrode guide comprises a planar disc 201 (FIG. 9C) having bores 202 therethrough spaced to accept electrodes of a given array, and a central injection needle. The central needle guide can provide an elongated portion 203 on the invention head component side of the planar disc 201 to aid the insertion of the injection needle and maintain the injection needle and electrodes at a 90 degree orientation with respect to the surface of the tissue being treated. For example, the electrode guide 200 can be placed against the tissue surface and the head portion of the invention device can be brought into proximity with the guide. Further connected with the guide, is an orientation component 204 which can fit into orientation slot 205 at the terminal end of the safety shield. The orientation guide 200 can thereby further detatchably connect to the safety shield by the slot 205 engaging the orientation component 204, or alternatively, upon inserting orientation component 204 into said slot 205, remain unengaged for immediate ability to become detached. The guide can further act as a penetration depth limiter by designing the disc guide with an appropriate thickness or by designing the elongated portion 203 to an appropriate length for the particular electrode/injection needle desired.

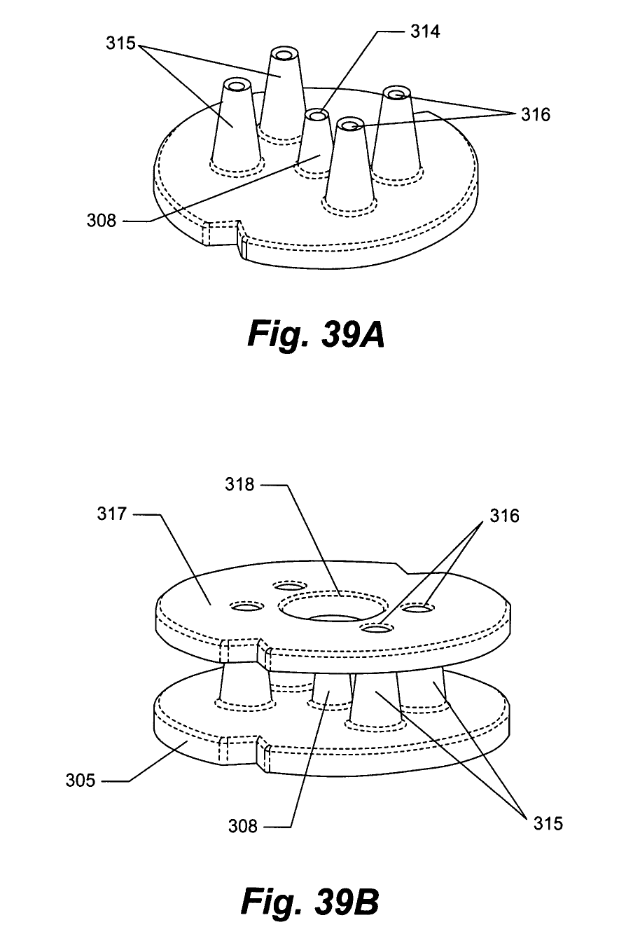

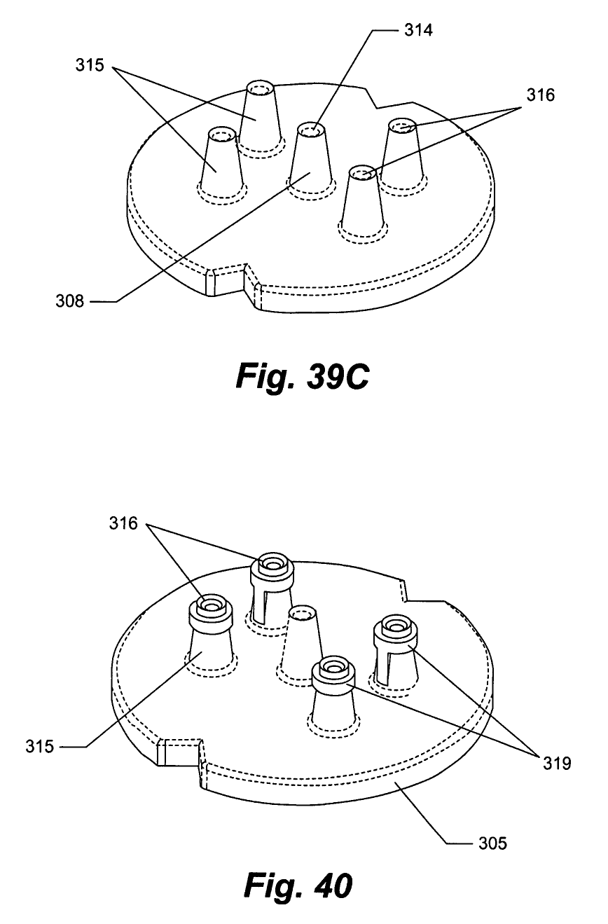

In alternative embodiments, the discoid guide can be designed with features as disclosed in FIGS. 34 to 42. For example, in a first embodiment, the guide can comprise a planar discoid substrate 305 with a first and second surface and a plurality of throughholes bored therethrough. As shown in FIG. 34, the electrodes of an electroporation device 300 having a head section 301 and removable electrode portion 302 comprising a plurality of needle electrodes 303, can be inserted into electrode guide bores 307 such that the electrode needles are guided into the tissue in a direction 90 degrees to the tissue surface and in a direction parallel to one another (since the guide maintains the needles in a predetermined parallel trajectory). In a related embodiment, the invention guide includes needle insertion orientation markers 306 which can be aligned with alignment markers 304 on the electroporation device. The markers can be constructed in any manner useful for notation of orientation so that the electrode needles of the electroporation device are aligned for insertion into the electrode bores of the invention guide. In a further related embodiment, the openings of the upper portions of the bores (whether injection needle bore or electrode bore) are funnel shaped (see FIG. 39A, element 314 for example) for easy insertion of the needles into the bores as they are guided into the depths of the bores and into the underlying tissue, the cone-shaped opening of the bores having a substantially larger diameter than the injection or electrode needles.

In another embodiment, as shown in FIG. 35, the invention guide can be used with an electroporation device that has needles oriented in different positions relative to the electroporation handle.

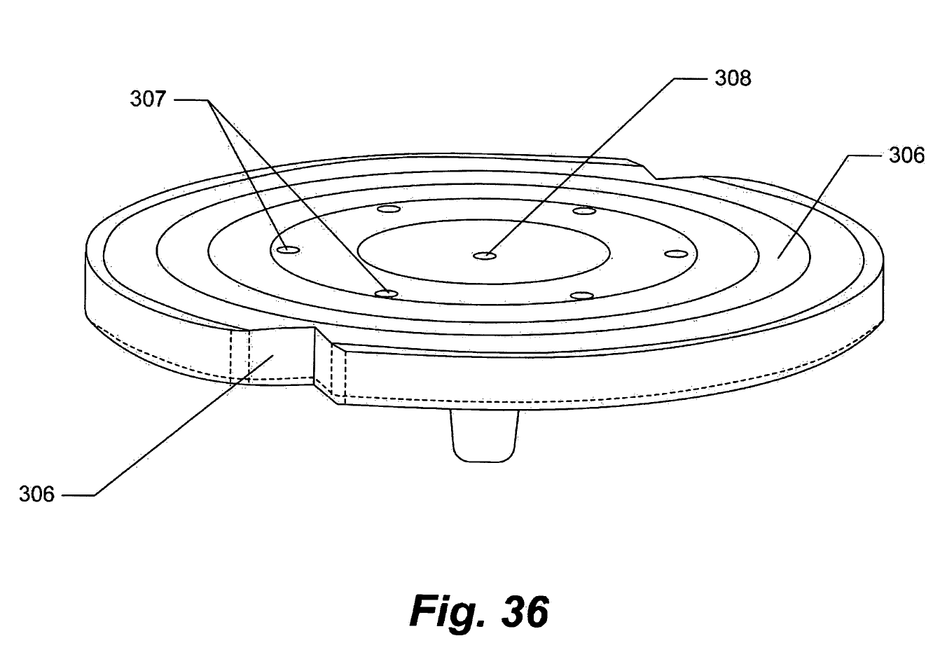

As shown in FIG. 36, the first side of the planar substrate of the invention guide has applied to selected portions thereof an adhesive material 310 consistent with adhesives used in other medical equipment wherein it is desired for the substrate to be maintained in one position relative to the tissue to which it is adhered. For example, adhesives used on medical tape, moleskin, band aides etc. are applicable as are easily understood by those of ordinary skill in the art. As shown, the electrode 307 and injection needle 308A bore openings on the first side are not expanded in diameter as are the upper end of the bore openings on the second side, but rather comprise open cylinders slightly larger than the electrodes or injection needle intended for a particular guide.

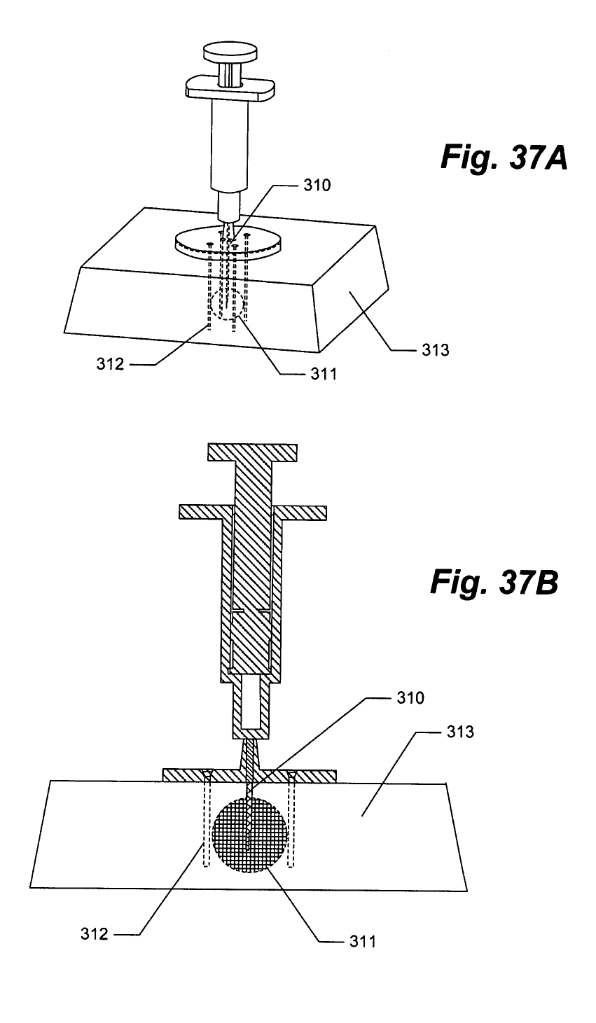

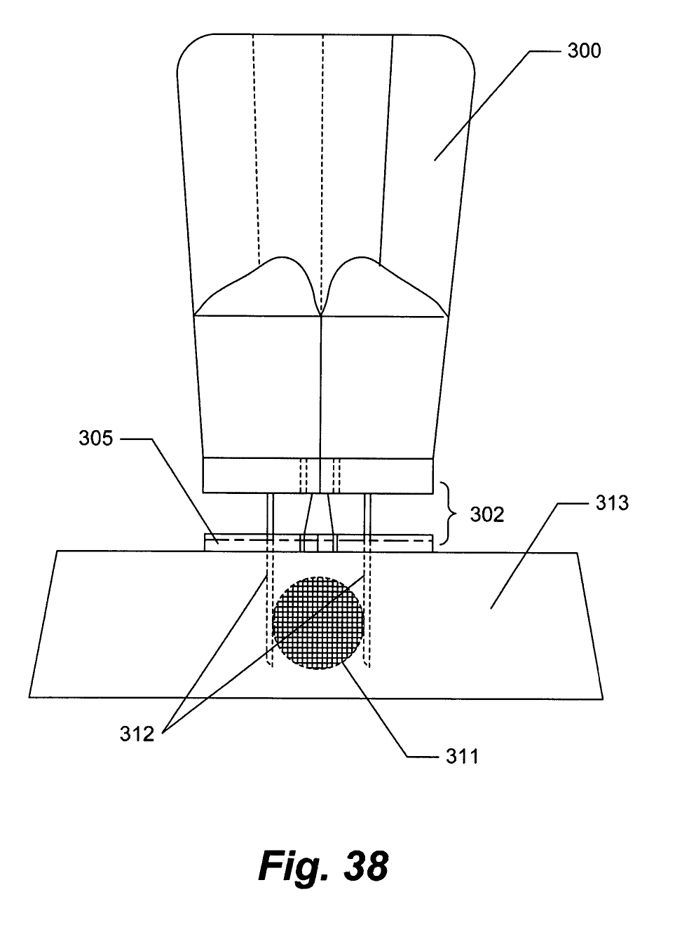

In use, the invention guide, as shown in FIG. 37A is first attached to a tissue surface. Next, an injection bolus is delivered by inserting a syringe needle 310 of a given length and diameter dimension into the central bore 308 of the invention guide; the needle being inserted to a depth limited by the height of the substrate surrounding the central bore 308 (See FIG. 38A).

After injection of the bolus 311, the syringe and needle are removed from the guide and an electroporation device is properly oriented over the guide using orientation markers such as markers 306, and the electrodes are inserted into the electrode guides. The invention guide can contain electrode bores for any number of electrodes but typically, the guide has a multiplicity of bores for a geometrically arranged array of electrodes. Preferably, the bores can be arranged in a square, rectangle, circle or oval, hexagon, octagon, etc. and can include at least two electrode bores, three electrode bores, or four, five, six, seven or eight bores. Additionally, the bores may be arranged with respect to one another in any dimension but preferably spaced between 0.2 and 2.0 cm apart from the nearest electrode of opposite polarity.

As shown in FIG. 38B, after the injection needle is removed and the electroporation needles inserted into the guide, the electrodes 312 are inserted to the depth allowed by the guide and the electrodes are then activated. In such operation, due to the presence of the guide, the tissue 313 being electroporated will be properly oriented relative to that tissue subjected to the injected bolus 311.

In further embodiments, the guide can support any number of electrode lengths and geometric configurations. As shown in FIG. 39A-C, other manifestations of the invention are possible. For example, in FIG. 39A, the substrate comprising the extensions making up the electrode guides 315 is taller than the substrate extension making up the central injection needle guide 308. In FIG. 39B, the electrode needle guides 315 are substantially longer than the injection needle substrate extension 308. In this embodiment, the upper portions of the electrode needle bores have connected therewith a donut shaped cowling 317 for support and for inclusion therewith of an orientation guide for easy viewing of orientation of the electroporation device to the guide. In FIG. 39C, yet another example of the invention guide is displayed wherein all of the electrode and injection needle bore extensions are of equal height.

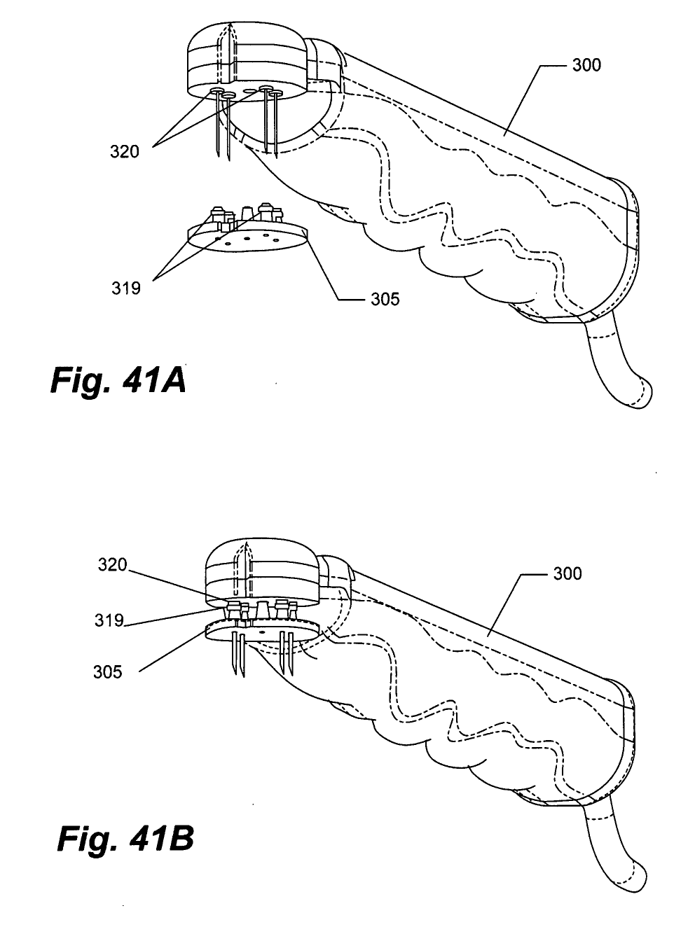

In a further related embodiment, the invention guide provides for a safety mechanism wherein the electrodes of the electroporation device cannot be pulsed with an electric signal unless the electrodes are “fully” inserted into the guide. In the first instance, full insertion ensures that the electric field generated by the electrodes is in proper orientation relative to the injected bolus. For example, as shown in FIG. 40, the tips of selected ones of the substrate extensions on the invention guide, whether injection needle or electrode needle, which also provide for depth limit or “stops” for the electrodes, have applied thereto an electrically conductive material 319 such that when the tip of the stop contacts the substrate in which the electrode needles of the electroporation device are mounted, an electric signal is completed between an anode and a cathode electrical contacts 320 placed on the electrode substrate near the needle electrode (see FIG. 41A). When fully inserted, the electrically conductive material 319 is in contact with electrical contacts 320 (see FIG. 41B). Alternatively, the cathode and anode contacts can be placed centrally between the needle electrodes on the electroporation device so that the electrically conductive material on the injection needle stop tip can close a circuit therebetween when the electroporation device is fully inserted into the invention guide.

In a further embodiment, the invention guide can include, integral with portions of the substrate on the side containing the electrode stops, additional engagement elements for engaging a safety shield of the electroporation device incorporating such a shield when the invention guide is properly aligned with the electroporation device for insertion of the electrodes into the guide bores. In this embodiment, proper engagement is required for the safety shield to retract and allow the needles to be inserted into the guide. For example, as shown in FIGS. 42A and B, the upper ends of the substrate surrounding the electrode or injection needle bores can abut elements on the safety shield that when contacted properly, as by properly being oriented, the shield will be allowed to retract.

In still further related embodiment, the direction guides and orientation guides provide for the capability of ensuring that the delivery of fluid medium through the injection needle remain in a bolus within an area central to the needle electrodes. Such capability provides for consistent delivery of a therapeutic agent to the proper orientation with respect to the positioning of the electroporation electrodes.

In a further embodiment, the safety shield can be limited in the amount of travel it is allowed to retract. Such limitation can be brought about by stop 64 (see FIG. 9A) which limits the movement of the safety shield to a predetermined distance which is set according to the length of the needle electrodes and injection needle extending from the injection port. More particularly, in one embodiment the stop 64 comprises the handle end of the safety shield which impinges on a portion 65 of the head which comprises connector 23.

Connected at the handle end of the central core support 21 is a safety shield guide 90 (see FIGS. 5A, and 7A). The safety shield guide 90 comprises a cylindrical tube sized in diameter greater than the diameter of the central core support 21 to the extent necessary to allow the passage between said central core support 21 and said guide 90 of the safety shield 50 as it is retracted to expose the electrodes. In a related embodiment, the closed channel 28 that is attached to the handle side 22 of the central core support 21 and leading to the syringe connector 29, extends beyond the diameter of the shield guide 90 and thereafter leads to the syringe connector in a direction away from the head and toward the handle component. FIGS. 5B and 6 show, for example, two orientations for the channel 28 and connector 29.

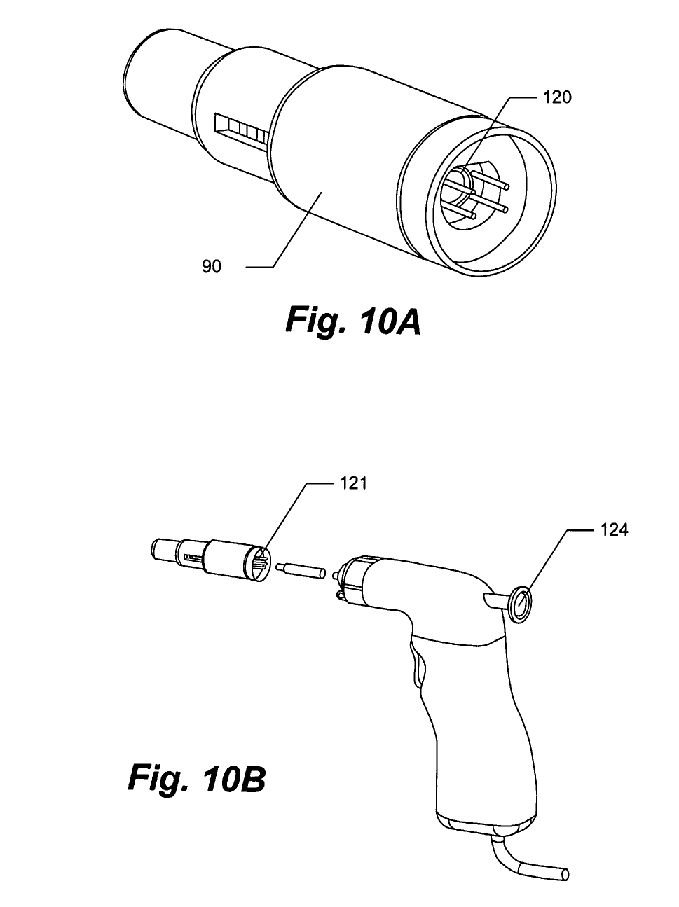

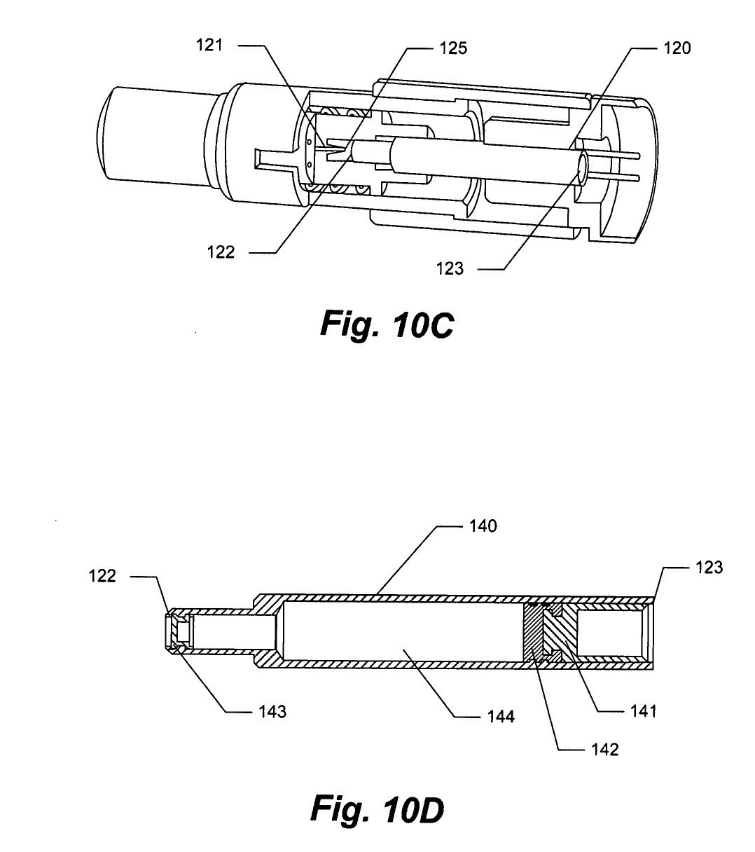

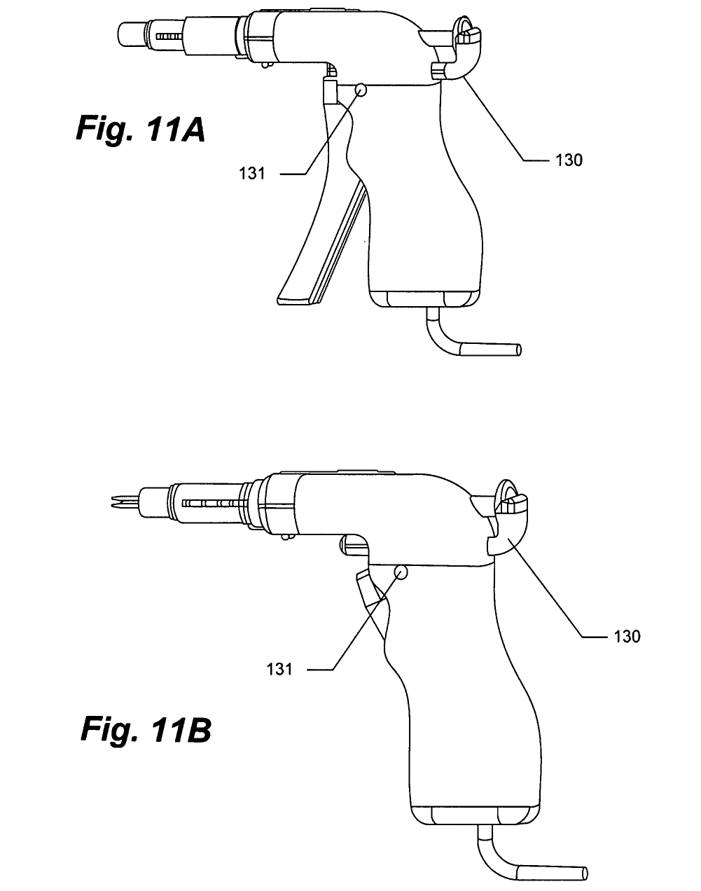

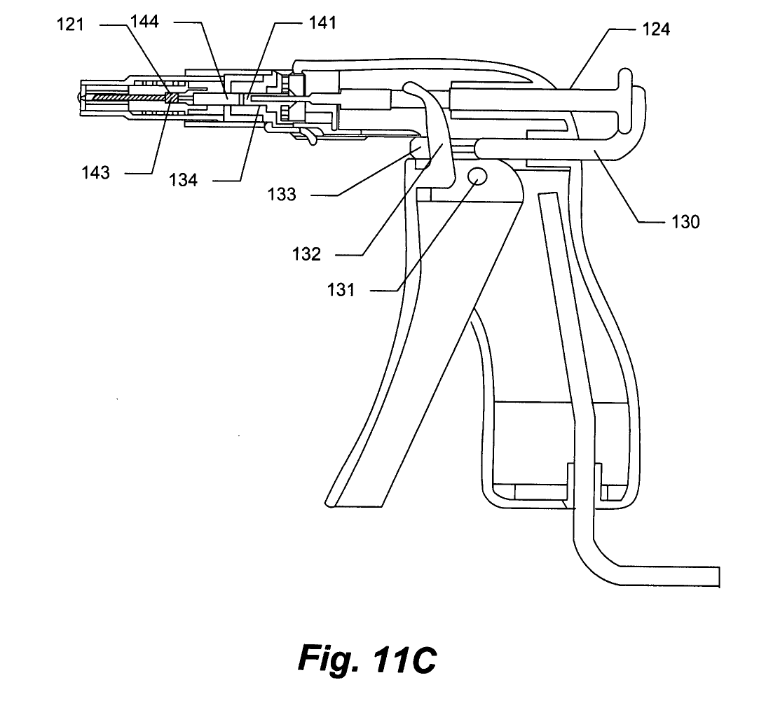

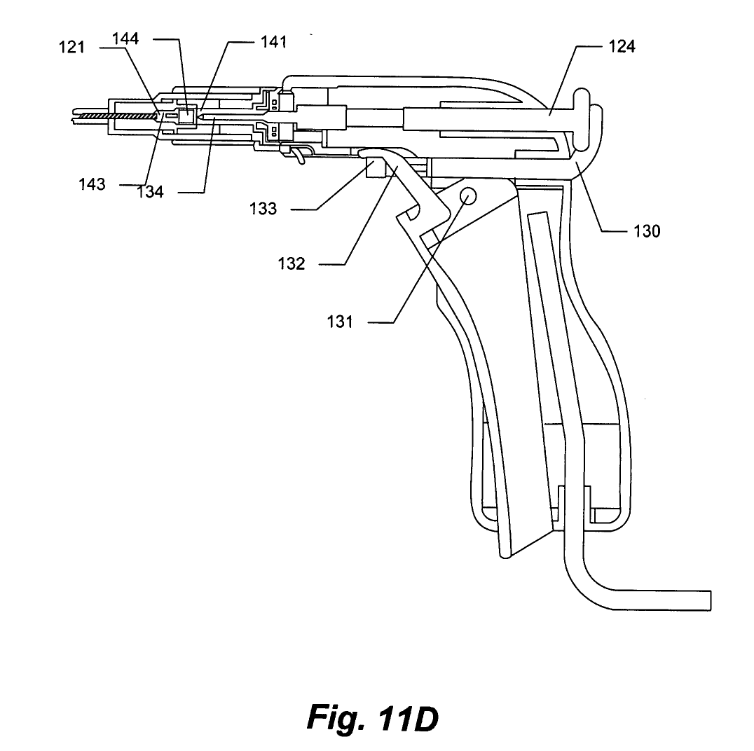

In another embodiment, as shown in FIG. 10A, the head component comprises a receptacle 120 within the handle side of the safety shield guide 90. The receptacle 120 comprises an open shaft forming a cylinder into which an appropriate sized cylindrical vial containing a medicament can be guided therethrough to a depth sufficient to encompass the vial as desired, with the end of the vial within the shaft abutting the handle side of the central core support as shown in FIG. 10C. In this embodiment the handle side of the central core support bore opening terminates in a sharp tubular canula 121 (which may be covered by a rubber seal 125 and said needle sufficient to pierce a rubber seal 122 on one end of said vial. In a further related embodiment, the end of the vial opposite the central core could comprise a sealably moveable piston 123 such that upon piercing the rubber seal 122, the handle end of the vial could be forcibly pushed into the cylinder of the vial. As depicted in FIG. 10D, a therapeutic-containing vial for use in association with the invention device can comprise distal end 122 with rubber seal 122 and vial septum 143, chamber 144 for containing a therapeutic fluid, plunger seal 142 and vial plunger 144, and finally piston 123. In a further related embodiment, the handle can be equipped with a plunger element 124 as depicted in FIGS. 10B and 11A-D.

The invention apparatus can include further embodiments including the capability of injecting a therapeutic agent from a syringe or a compressible vial by a fully- or semi-automated delivery and electrode activation system. For example, the handle can include a lever or other appropriate mechanical tensioner 130 designed to connect to the plunger of a syringe or a plunger built into the handle for pressing against the piston of a therapeutic containing vial such as depicted in FIGS. 11A-D. In such embodiment, the action of squeezing the trigger causes the plunger of either the syringe, or a plunger integral with the handle to force fluids out of the syringe or out of a vial and upon reaching a terminal point to which the trigger is squeezed, the electrode activation switch is activated and the electrodes are energized for electroporating the cells of the treated tissue. Alternatively, the mechanical squeezing of the lever arm to activate the tensioner can be replaced by an electronic actuator which moves the tensioner and following deployment of a fluid therapeutic agent from the syringe or vial, activates (i.e., energizes) the electrodes. For example, as shown in FIGS. 11C and 11D, squeezing the trigger can cause a cam 132 to engage the end 133 of actuator/tensioner 130 causing the actuator 130 to force the plunger 124 into a vial or alternatively, force a syringe plunger into a syringe.

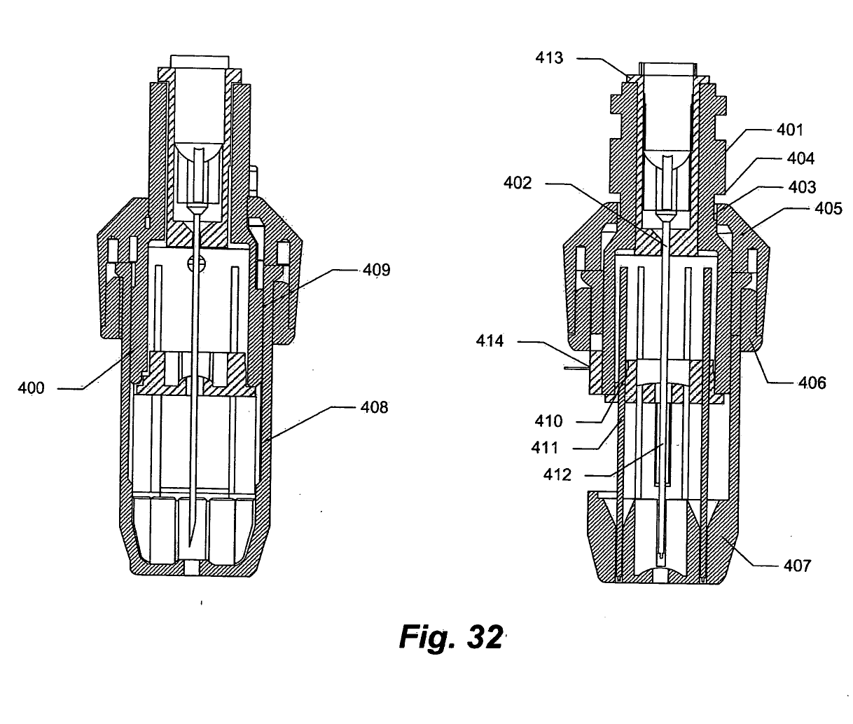

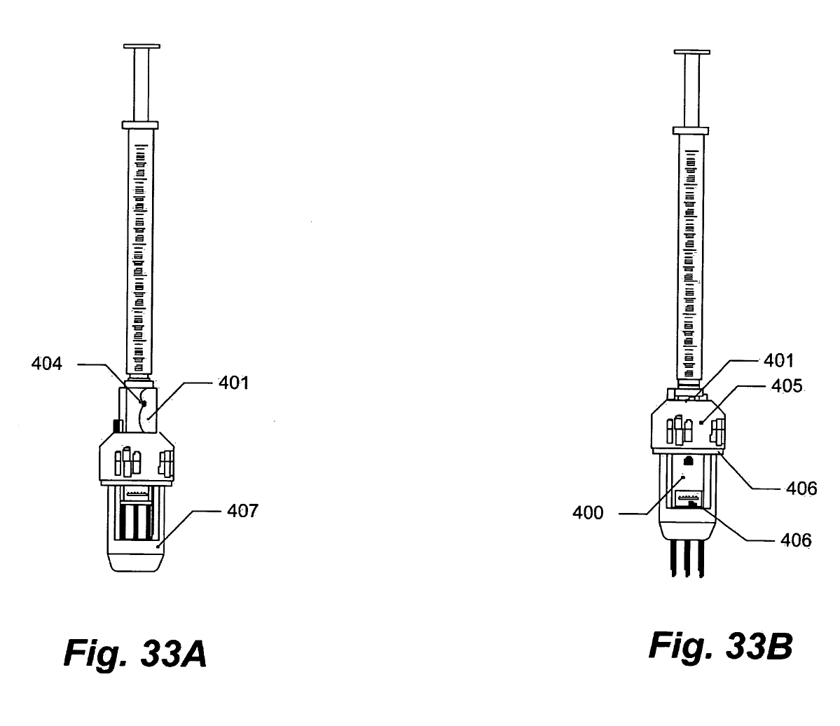

In still further alternate embodiments, the head component can comprise means to connect directly to a hypodermic syringe as depicted in FIGS. 32 and 33. In this embodiment, the head component assembly comprises a minimal number of parts including a plurality of elongate electroporation electrodes mounted in a plug substrate 410 in a substantially geometric pattern in spaced relation to one another and along and/or within a predetermined circumference defined by the dimensions of the plug substrate 410 and about a bore 402 centrally placed through an electrode substrate 400 to which the plug substrate 410 attaches. The bore 402 is open on two sides of said substrate. Preferably, the elongate electrodes extend from a second side of said substrate for a predetermined distance, and on a first side of said substrate there are no electrodes extending therefrom. In a preferred embodiment, the bore has a central axis parallel with the linear axis of said plurality of elongate electrodes, all of which are parallel with respect to one another.

On said first side of said substrate, i.e., the side opposite said second side, is a hypodermic syringe connector means 413. Said connector means 413 can be of any useful design for attaching the expulsion nipple of a typical syringe and/or hub of a hypodermic needle attached thereto to an extension 401 of the electrode substrate 400. The connector further comprises a bore for inserting said syringe nipple wherein said connector bore is in an open channel alignment with the bore of said substrate.

In another embodiment, the substrate is slidably engaged with a translucent or clear substantially cylindrical housing forming a safety shield 407. The shield and substrate are kept in place relative to one another by a locking cap 405 and keeper 406. The locking cap 405 is capable of locking the substrate 400 in a first and/or a second position relative to the shield by a rotation-based locking pin 403 and pin guide 404. Additionally, the substrate is kept from rotating within the translucent safety shield 407 by slide guides 408 comprising channels formed into the walls of the shield 407 and corresponding slide tabs 409 formed at the circumference of the substrate 400.

In other embodiments, the syringe connector 413 can comprise a syringe “snap” that keeps the syringe from disengaging the syringe connector.

In still another embodiment the safety shield 407 can include a needle guide at its end as described earlier herein comprising a plurality of bores, one for each electrode and hypodermic needle to assist the parallel trajectory of the electrodes and needle as they are forced into the patient tissue. Further still, the bores can be conical shaped to allow easy entrance of the electrode and needle tips into the guide bores.

In operation of the embodiment comprising a head that is directly connectable to a syringe, a user will prepare a syringe and needle with a volume of fluid containing a medicament. The electrical connector port 414 will be attached to a plug and wire leading to a source of electrical energy. The syringe will be inserted into the substrate bore 402 so that the nipple/needle butt removably connects with the syringe connector 413 on the substrate 400. This is followed by the user rotating the locking cap 405 to the “unlocked” position so that the substrate 400 can slide. The electrode assembly is then placed at the treatment zone on the patient tissue and the needles are slid through the guide bores in the end of the shield so as to allow the needles to penetrate into the patient tissue. The user can then inject the material from the syringe. If desired, the locking cap 405 can be rotated to lock the device in the “open” position with the needles still in the patient subject. If desired, the hypodermic needle can be removed from the assembly and the electrodes activated without the hypodermic being in the field of electric energy imparted by pulsing the electrodes. Alternatively, the hypodermic needle can be left in the field while the electrodes are pulsed. Finally, the assembly can be removed from the patient tissue and discarded.

The invention device has industrial applicability and use by veterminarians, biomedical researchers or in the clinic or office or in the field by physicians and provide for various manners of carrying out electroporation of patient tissues. For example, where non-penetrating or semi-penetrating electrodes are used, treatment may be carried out for local surface treatment by transcutaneous electroporation, i.e., the electric fields imparted by the pulse generator's energizing of the electrodes are generated external to the tissue to be treated although part of the electrical field must penetrate into the target tissue in order to effect electroporation. Generally, surface ailments, such as shallow lesions of cutaneous head and neck cancer and melanomas are accessible and subject to treatment using such electroporation method. Alternatively, where tissue penetrating electrodes are used, the electric field is generated within the tissue to be treated. Generally, treatment by generation of the electric filed internally allows for electroporation of subcutaneous and muscle and internal organ tissue.

In preferred embodiments, the device is useful for electroporation-based delivery of drugs for treating cancers, and for gene therapy. For example, uses for the invention include treatment of any ailment, condition or disease with DNA, RNA, or oligonucleotides of any composition and structure. Further, the device is useful for delivering vaccines comprising either protein or expressible nucleic acid constructs encoding proteins that are either active in a biologic, including metabolic, process or comprise antigens for generating an immune response. In such embodiments of use, the substances electroporated to a patient tissue are generally therapeutic agents. As used herein, a therapeutic agent can comprise a nucleic acid encoding a polypeptide or another nucleic acid e.g., for example, an RNA molecule, said nucleic acid encoding a polypeptide or other nucleic acid capable of expressing the peptide or the other nucleic acid encoded thereby in biologic cells. A therapeutic agent can further comprise drugs, small molecules, lipid bound molecules or anti cancer compounds.

In further embodiments, the outcome of gene expression level and degree of immunological activation in a subject tissue can be predetermined, generally, by the Voltage level applied, the dimensions of the electrode array chosen, and the pulse conditions, (i.e., number of pulses and duration of pulse).

As with other methods used for medical or veterinary treatments, or in medical research endeavors aimed at the development of such treatments, the two factors of greatest concern in the use of electroporation are safety and efficacy. The efficacy of electroporation in delivering a large variety of drugs and biological molecules, including nucleic acids and proteins, into biological cells has been extensively studied and documented. On the other hand, the safety and side-effects of electroporation have been studied to a much lesser extent and are less well understood. Known side-effects of electroporation include pain, muscle contractions, erythema and burns, the latter only in rare cases when surface electrodes were used. Histological effects of electroporation that vary with the particular target tissue include apoptosis, necrosis, inflammation, fibrosis and mild hemorrhage. Target tissues frequently subjected to electroporation for purposes of treatment or for the development of new treatments include solid tumors, muscle and skin. While tumors have been treated extensively with chemotherapeutic drugs delivered directly to tumor cells by electroporation, normal muscle and skin tissue have been of particular interest as target tissues for gene therapy and DNA vaccination. For cancer gene therapy DNA has also been injected intratumorally in some animal experiments. In therapeutic applications, the goal is to minimize side effects while maximizing the therapeutic effects. One of the potentially most serious side effects in gene therapy is the triggering of an immune response against the transgene product, which not only may render the therapeutic transgene product ineffective, but could also be life-threatening to the patient, or prevent future therapy with the “recombinant” therapeutic gene product manufactured in cell culture due to sensitization of the immune system to said product, for example.

As mentioned, one of the side effects caused by electroporation is local tissue inflammation, which enhances anti-transgene immune responses generated as a result of the inflammatory response. Therefore, for gene therapy applications, inflammation needs to be minimized. On the other hand, some degree of inflammation is actually desirable in DNA vaccination applications to increase the efficacy of the immune response. In determining the parameters responsible for the severity of the inflammatory response, we found we were able to customize the inflammatory response elicited by electroporation depending on the intended purpose. For example, in using electroporation of muscle tissue for the delivery of DNA encoding the blood clotting agent Factor IX, for the treatment of hemophilia B, inflammation needs to be minimized. Conversely, for vaccination with a potent antigen, a low degree of inflammation may be optimal, while for a less potent antigen a higher degree of inflammation may be desirable.

In a study described in Example 3, experiment 1, various numbers of pulses of various voltages and field strengths were delivered with 6-needle arrays inserted percutaneously into muscle tissue of pigs. Prior to electroporation, the treatment site was either injected with saline or the anticancer drug bleomycin. Histological changes of the treated tissues were evaluated on day one after treatment and at different intervals up to 40 days post treatment. The results are presented extensively in Example 3. Table 5, section A, highlights a small portion of those data which illustrate an important finding of this study. Three different sized needle electrode arrays (i.e., 0.5, 1.0, and 1.35 cm diameter) were energized with different voltages (560 to 1500 V) resulting in similar minimal field strengths (approximately 1111 to 1302 V/cm). Whereas the application of 560 V and 1130 V did not result in significant muscle inflammation under the experimental conditions, the application of 1500 V did clearly evoke muscle inflammation. We concluded from this result that the applied voltage, or a factor related to the applied voltage is a determinant for the degree of histological changes induced by electroporation, and that field strength within the ranges tested is not a determinant. This conclusion, which is consistent with the other data presented in Example 3, is important because the effectiveness of the electroporation event, i.e., causing the cells to become porous to the injected therapeutic agent, depends on the field strength and not on the applied voltage. However, a certain minimal field strength (threshold value) is required for electroporation to occur). Thus, separating the effect of the field strength (i.e., electroporation efficiency) from the effect of the voltage, or voltage related function (i.e., histological change including inflammation), the effect of the field strength which relates to electroporation efficiency. In deciphering the distinction between Voltage and field strength we are able to manipulate, within limits, the degree of histological changes and inflammation, while maintaining high-efficiency electroporation and therefore higher levels of gene expression.

In Table 5, section B, selected data of Example 3, experiment 2, are presented. Animals were injected intramuscularly with DNAs coding for two antigens, glycoprotein D of BHV-1 and HBVs Ag. After injection, the treatment sites were electroporated using either 100V or 200 V, with all other experimental parameters kept constant. The animals electroporated with the lower voltage displayed a lower degree of muscle inflammation than the animals electroporated with the higher voltage. The immune response to glycoprotein D, a potent antigen, was greater with the higher voltage treatment than with the lower voltage treatment, although the levels of antigen expression were similar in both cases. This finding supports the conclusion and prediction derived from the data in section A of Table 5 and Example 3, experiment 1, namely, that higher voltages cause greater inflammation and greater stimulation of the immune response than lower voltages.

TABLE 5
Influence of electroporation pulse parameters on histological changes

| | | | | | | | | Gene | |
|---|---|---|---|---|---|---|---|---|---|
| | Agent | Voltage | Nominal Field | Number | Pulse | Nominal Energy | Electrode | Expression | Histological Change |
| Section | Injected | (V) | Strength (V/cm) | of Pulses | Length | Delivered (J) | Array | Level | (muscle inflammation) |
| A | Saline | 560 | 1302 | 6 | 100 usec | 1.9 | 6-NA, | n.d. | None on days 1, 5 |
| | | | | | | | 0.5 cm | | |
| | Saline | 1130 | 1314 | 6 | 100 usec | 7.7 | 6-NA, | n.d. | None on days 1, 5 |
| | | | | | | | 1.0 cm | | |
| | Saline | 1500 | 1111 | 6 | 100 usec | 13.5 | 6-NA, | n.d. | Score 2 on day 1; |
| | | | | | | | 1.35 cm | | Score 3 on day 5 |
| B | DNA in PBS | 100 | 116 | 2 | 60 msec | 12 | 4-NA, | High | Moderate to |
| | | | | | | | 1.0 cm | | severe on day 2 |
| | DNA in PBS | 200 | 233 | 2 | 60 msec | 48 | 4-NA, | High | Severe on day 2 |
| | | | | | | | 1.0 cm | | |
| C | DNA in PBS | 100 | 233 | 2 | 60 msec | 12 | 4-NA, | High | n.d. |
| | | | | | | | 0.5 cm | | |
| | DNA in PBS | 200 | 233 | 2 | 60 msec | 48 | 4-NA, | High | n.d. |
| | | | | | | | 1.0 cm | | |

n.d. = not done

Further, section C of Table 5 discloses data described more extensively in Example 1. Gene expression was determined using two needle array electrodes of 0.5 and 1.0 cm diameter, respectively, and applying voltages of 100 and 200 V, respectively, which resulted in equal nominal field strengths of 233 V/cm in both cases. All other experimental parameters were kept constant. The result shows that under both electroporation conditions essentially the same level of gene expression was obtained. This finding supports the notion that at equal field strengths, electroporation efficiency is the same, within limits, although the applied voltage differed by a factor of two. As stated above, the applied voltage, or a function related to the applied voltage, appears to be the main determinant of the degree of histological changes, including inflammation. When the amount of energy of various electroporation pulses is calculated using the formula:
W=V²/R×t×N,
where W is the energy in Joules (J), V is the voltage applied in Volts, R is the resistance of the tissue in Ohms, t is the duration of the pulse in seconds, and N is the number of pulses applied, the correlation of the degree of histological changes, including inflammation, with the amount of energy applied, is even better than the correlation with the voltage (Table 5, sections A and B). Thus, the pulse duration and pulse number also influence the degree of histological changes, although only in a linear fashion, whereas the energy delivered, and the histological changes elicited, increase with the square of the applied voltage. Therefore, voltage appears to be the most important but not the sole factor to control when histological changes and inflammation are to be manipulated. Correspondingly, the invention device further allows for its use in a predetermined outcome of the level of gene expression, the level of inflammatory response, and the strength of the immune response by choosing appropriate electropoation pulse parameters. The field strength determines electroporation efficiency and thereby the uptake of the agent into the cell, and thus, eventually, the effect of the agent within the cell (e.g., cytotoxicity in the case of an anticancer drug, and gene expression in the case of DNA, respectively). The energy delivered determines the degree of histological changes, including inflammation, which strongly influences the effectiveness of the immune response (e.g., enhancement of immunological anti-tumor responses in the case of tumor electroporation, and anti-transgene product responses in the case of gene delivery into muscle).

Further, field strength at constant voltage can be manipulated by sizing the electrode appropriately. For example, if a low field strength and high voltage is desired, the distance between negative and positive electrodes will be relatively long. Delivered energy can be manipulated by adjusting voltage, pulse length and number of pulses at a given tissue resistance. The same amount of energy can be delivered by pulses of different voltage, number, and duration. The choice of these parameters also influences electroporation efficiency and histological changes. For example, for the delivery of DNA, pulse durations of less than approximately 10 milliseconds are relatively inefficient, whereas short pulses in the range of 0.1 milliseconds are effective in the delivery of low molecular weight drugs and small peptides.

Other factors which also may play roles of various importance in manipulating electroporation efficiency and histological changes include, but are not limited to, needle electrode diameter, pulse frequency, electrode surface composition and the structure, composition and electrical conductivity of the target tissue. For example, pulse frequency (i.e., the time interval between individual pulses) influences the degree of changes occurring in the interstitial space and in cells close to the electrode surface. The changes are greater when pulses are delivered at high frequency, presumably because the tissue is given less time to recover between pulses. For example, we have observed that the electrical current that flows between needle electrodes inserted into muscle decreases significantly with every subsequent pulse if the frequency is relatively high (e.g., about 4 Hz), but decreases to a much lesser extent when the frequency is low (e.g., about 0.5 Hz or less).

Utility of an electroporation device such as the current invention is demonstrated by the following Examples which exhibit that elements of the invention device provide for efficient and patent friendly outcome.

### EXAMPLE 1

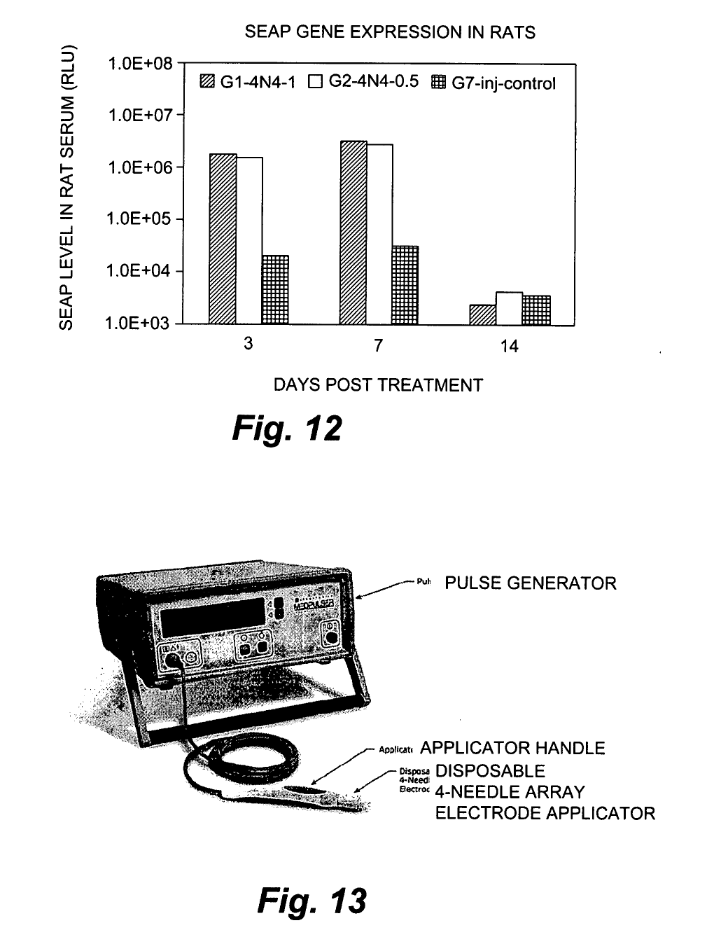

The electrode array can have a diameter of between 0.2 and 2.0 cm. With respect to electrode arrays having either a 0.5 or a 1.0 cm diameter, an experiment was performed showing that a nucleic acid sequence encoding secreted alkaline phosphotase (SEAP), following injection of said nucleic acid and electroporation, was expressed equally in the treated tissue of subject rat muscle using either 0.5 cm or 1.0 cm electrode arrays at equal nominal field strengths. (See FIG. 12).

Experimental conditions:

Sprague Dawley rats (n=5 per group)
Bilateral intramuscular injection in the hind tibialis anterior muscles (50 μg pSEAP in 100 μl/leg)
EP settings: For 0.5 cm 4-needle array: 100V, 60 ms, 2 pulses
   For 1.0 cm 4-needle array: 200V, 60 ms, 2 pulses
      Brief summary of the results:
EP groups (0.5 cm array and 1.0 cm array) demonstrate about two orders of magnitude increase in SEAP expression in the serum compared to the control group (Dna Injection without Ep)
The efficacy of the 0.5 cm array is equivalent to the 1.0 cm array in terms of SEAP level over the control, e.g., on day 7, there is a 97-fold enhancement for the 1.0 cm array over the control and a 92-fold enhancement for the 0.5 cm array over the control.
The difference between the SEAP level obtained with the 0.5 cm array and the 1.0 cm array, respectively, is not statistically significant (P>0.05). However, the difference between the SEAP levels obtained with the EP groups and the control group, respectively on day 3 and 7, is statistically significant (P<0.01).

These results show that electroporation can be performed with electrode arrays comprising electrodes in an array, and injection of a substance, particularly a nucleic acid encoding a gene, between the electrodes of the array, as described for the invention modular head component, thereby providing substantial enhancement of uptake of said substance into the cells and resulting in enhanced gene expression (approximately higher than 90 fold over control).

### EXAMPLE 2

In this example, an experiment was performed to test whether an electroporation device, using electrodes and pulse parameters of which the invention device is capable, can be used on a patient without an anesthetic. For example, electroporation-mediated drug delivery to tumors is commonly performed under general anesthesia (e.g., for head and neck tumors) or local anesthesia (e.g., for cutaneous malignancies). Because it is important for a patient to be able to tell their physician of adverse affects caused by any given treatment, which ability would be impaired under anesthesia, and because in an out-patient setting such as in a physicians office, vaccinations should be performed quickly and easily as well as safely for the patient, anesthesia should be avoided, if possible. This is particularly important for DNA vaccination and gene therapy applications where repeated administration of DNA may be necessary to sustain gene expression. However, it was unknown whether EP of muscle tissue, without anesthesia, is tolerable and safe in humans. To evaluate the safety and the pain sensation associated with EP of muscle tissue using needle electrodes we initiated a study in healthy volunteers to evaluate the pain associated with EP to muscle tissue.

Subjects

Five healthy adult men, ranging from 40 to 62 (mean 50) years of age, participated in the study.

Study Design

Screening and Enrollment. Subjects were screened for eligibility to participate in the study. During the screening visit (day −14 to 0), subjects' baseline vital signs (temperature, blood pressure, pulse, and respiration) were measured, and medical history and concurrent medications were recorded.

Electroporation Device. Electroporation of the muscle was performed using a pulse generator and a linear handle type apparatus as shown in FIG. 13. In the study, only the sensation associated with the electroporation pulsing of needle electrodes was of concern, therefore the apparatus did not include the embodiment of a syringe holder. The apparatus comprised 1) the pulse generator; 2) an applicator handle; and 3) a disposable head component having four 1 cm, 26-gauge gold-plated stainless steel needles inserted into a central core support. Two sizes of needle electrode head elements were used, namely a 0.5 cm diameter four needle array comprising needle electrodes at the corners of a 0.25×0.43 cm rectangle inscribed in a circle of 0.5 cm diameter, and a 1.0 cm diameter four needle array comprising needle electrodes at the corners of a 0.5×0.86 cm rectangle inscribed in a 1.0 cm circle. The head elements/electrodes were sterilized using ethylene oxide and disposed of after one-time use. The pulse parameters (voltage, pulse length, number and frequency of pulses) were pre-programmed, and pulses were recorded using an oscilloscope to verify the system performance.

Procedures. Procedures were administered at Day 1. Two procedures were administered to each subject (Table 1). No anesthetic, drug, or nucleic acid was administered in either procedure.

TABLE 1

| | | 4-Needle | |
|---|---|---|---|
| Procedure | Deltoid muscle | array | Settings of MedPulser ® DDS* |
| 1 | Left arm | 0.5-cm | 100 V, 60 ms, 2 pulses |
| 2 | Right arm | 1.0-cm | 200 V, 60 ms, 2 pulses |

*DDS = DNA Delivery System

Procedures were performed and results recorded as follows: Procedure 1 was initiated by cleaning the skin at the test site with isopropyl alcohol. A 0.5 cm 4-NA was inserted percutaneously into the deltoid muscle of the left arm. The pain score associated with the insertion of the array was determined by the subject and was recorded immediately. Then, two 60-ms electrical pulses of 100 V were delivered at 4 Hz using the pulse generator. The pain score was determined by the subject and was recorded immediately. Any physical response to either the insertion of the array or the EP pulses was recorded by the physician. Procedure 2 was administered only if Procedure 1 was tolerated, as determined by the subject. Procedure 2 was analogous to Procedure 1, except that the array was a 1.0 cm sized array and was inserted into the deltoid muscle of the right arm and the voltage of the pulses was 200 V.

Post-Procedure Assessment. Subjects were required to visit the investigator's office 24 hours and 30 days after the procedures to monitor any local and/or systemic adverse reaction and to monitor potential long-term pain.

Endpoint Measurements

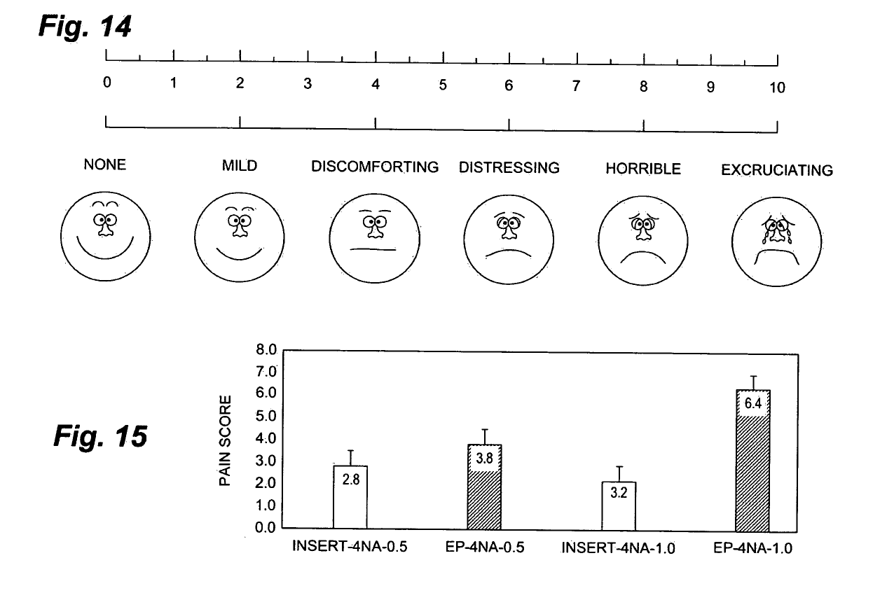

Pain Assessment. Pain scores were measured using a numeric scale consisting of a 10 cm line with “no pain” written at one end and the “worst imaginable pain” written at the other end (FIG. 14). Before the procedures, the subject was asked to review both the visual and numeric pain scales. Upon completion of each procedure, a pain score was given by the subject and recorded immediately.

Safety Assessment. The pain at the EP site and any local and/or systemic adverse reaction were monitored by the physician or nurse on Day 1, as well as 24 hours and 30 days post-procedures. Pain scores and adverse events, if any, were to be recorded in the Case Report Form.

Pain Assessment. Pain scores for each procedure were plotted as the mean ±standard error of the mean (SEM). Due to the limited number of subjects, statistical analysis was performed using the Student's t-test to compare the pain scores from different procedures.

Safety Assessment. All subjects were included in the safety analysis. Adverse events, if any, were to be graded according to the NCI Common Terminology Criteria for Adverse Events v3.0. There was no local nor systemic adverse event and no pain reported by any of the subjects 24 hours and 30 days after the procedures.

Results

Pain Scores

The pain scores determined by the subjects are summarized in Table 2 and FIG. 15.

TABLE 2

| Subject | Pain scores | | | |
|---|---|---|---|---|
| No. | Insert-4NA-0.5 | EP-4NA-0.5 | Insert-4NA-1.0 | EP-4NA-1.0 |
| 1 | 5 | 5 | 5 | 8 |
| 2 | 3 | 5 | 3 | 7 |
| 3 | 2 | 4 | 2 | 6 |
| 4 | 2 | 3 | 2 | 4 |
| 5 | 2 | 2 | 4 | 7 |
| Mean | 2.8 | 3.8** | 3.2* | 6.4 |

*p = 0.004, one-tail and paired t-test comparing EP-4NA-1.0 and Insert-4NA-1.0.
**p = 0.01, one-tail and unpaired t-test comparing EP-4NA-1.0 and EP-4NA-0.5.

In 4 out of the 5 subjects, insertion of the 0.5 cm or the 1.0 cm needle electrodes into the muscle caused only mild or discomforting pain, while one subject found the pain somewhat distressing (score 5). Delivery of the 100 V pulses through the 0.5 cm array caused mild pain in one subject, discomforting pain in 2 subjects, and somewhat distressing pain in 2 other subjects. Pulses of 200 V delivered through the 1.0 cm 4-NA elicited discomforting pain in one subject and distressing pain in another subject, while 3 subjects rated the pain as horrible. Thus, while the insertion of either needle array and the delivery of 100 V pulses via the 0.5 cm 4-NA were given mean pain scores ranging from 2.8 to 3.8 (mild to discomforting), the delivery of 200 V pulses via the 1.0 cm 4-NA resulted in a mean pain score of 6.4. The mean pain score for EP via the 1.0 cm 4-NA at 200 V was the highest among all procedures. The difference between the mean pain score related to the 1 cm array (200 V) and the score related to the 0.5 cm array (100 V) is significant (P<0.01). The difference between the mean pain score related to the electroporation with the 1 cm array (200 V) and the score related to the insertion of 1 cm array is also significant (P<0.004).

These results showed that, overall, EP settings for DNA delivery to the muscle using a needle array is safe (no adverse events) and tolerable to subjects when administered without anesthesia. The study also shows that 100 V pulses delivered via a 0.5 cm array causes less pain than 200 V pulses delivered via a 1.0 cm array. These results are in agreement with the fact that voltage (and therefore current) influences the sensation of pain elicited by electric stimuli: as the voltage increases (and current too), pain also increases. However, keeping with the benefits of the current invention, the range approximately of 0.3 to 2.0 cm diameter arrays are capable of application in a clinical setting.

Additionally, whereas strong muscle contractions have been observed during EP-mediated drug delivery to internal tumors in patients under any of general anesthesia, conscious sedation, or local anesthesia, in this study, without using anesthesia, we only observed minor muscle twitches (no limb movement), which were neither disturbing to the subjects nor interfering with the procedures. The difference is almost certainly due to the different voltages applied, i.e., 100 or 200 V in this study, versus 500 to 1500 V for intratumoral drug delivery.

### EXAMPLE 3

In accordance with embodiments of the invention, the dimensions of the electrode array, particularly needle electrode arrays, when used in concert with pulsing parameters comprising particular voltages, allows for electroporation of body tissues at lower voltages (V) while maintaining field strengths (V/cm) that are higher and that additionally provide for an effective level of tissue/immune stimulation not possible at low voltages. In other words, as mentioned above, where voltages used are high, body tissues may be adversely affected such that there could be over stimulation of the tissue leading to damage caused by inflammatory immune reactions induced by the electroporation pulse at the site of electroporation.

In one aspect, needle electrode arrays having a diameter, or distance between the electrodes of about 0.5 cm and used in electroporation of patient tissue at a voltage of equal to or greater than 100 V, results in an effective electric field strength of 200+V/cm. By keeping the applied voltage low, e.g., lower than about 150 Volts, tissue damage can be kept low yet the field strength can remain high (V/cm) without an appreciable detrimental effect on the tissue. In this aspect, therefore, voltage levels can be manipulated easily to bring about a predetermined field strength and tissue damage combination that is predeterminable for programming into the electroporation scheme the degree of tissue damage desired for a directly related level of immune response.

For the immediate example, a study was performed to evalulate the toxicity and side effects of EP on normal porcine skin and underlying skeletal muscles using different voltages and electrode arrays of different dimensions.

### EXPERIMENT 1

Histopathologic Examination. Section of skin and underlying skeletal muscles were collected and processed by routine histologic techniques and stained with Hematoxylin and Eosin. Each sample was evaluated for the following histopathologic changes: Muscle necrosis, muscle inflammation, muscle hemorrhage, muscle fibrosis, epidermal damage, epidermal inflammation, and subcutaneous inflammation. Each of these were scored by severity and/or extensiveness scores as follows:

0-non-existent, 1-minimal, 2-mild, 3-moderate, 4-severe, and 5-very severe.

Muscle necrosis: Score 5 was given when all cells in most fields in the section examined were in advanced necrosis. Score 4 was given when most cells in numerous fields in the section examined were in advanced necrosis. Score 3 was given when many cells in some fields in the section examined were in early to advanced necrosis. Score 2 was given when some cells in a few fields in the section examined were in early to advanced necrosis. Score 1 was given when a few cells in a rare field in the section examined were in early necrosis. Score 0 was given when no necrosis was found in the section.

Muscle inflammation and subcutaneous inflammation: Score 5 was given when numerous, densely packed inflammatory cells were found in most fields in the section examined. Score 4 was given when numerous, inflammatory cells were found in many fields in the section examined. Score 3 was given when many inflammatory cells were found in some of the fields in the section examined. Score 2 was given when inflammatory cells were found in a few of the fields in the section examined. Score 1 was given when inflammatory cells were found in a rare field in the section examined. Score 0 was given when no inflammatory cells were found in the section.

Muscle hemorrhage: Score 5 was given when numerous, densely packed extravasated red blood cells were found in most fields in the section examined. Score 4 given when numerous extravasated red blood cells were found in many fields in the section examined. Score 3 was given when many extravasated red blood cells were found in some of the fields examined. Score 2 was given when some extravasated red blood cells were found in a few of the fields examined. Score 1 was given when a few extravasated red blood cells were found in a rare field in the section examined. Score 0 was given when no extravasated red blood cells were found in the section.

Musclefibrosis: Score 5 was given when 70-100% of the muscle tissue was replaced by granulation tissue or mature fibrous in most fields in the section examined. Score 4 was given when 50-69% of the muscle tissue was replaced by granulation tissue or mature fibrous in many fields in the section examined. Score 3 was given when 30-49% of the muscle tissue was replaced by granulation tissue or mature fibrous in some fields in the section examined. Score 2 was given when 1-29% of the muscle tissue was replaced by granulation tissue or mature fibrous in a few of the fields examined. Score 1 was given when less than 1% of the muscle tissue was replaced by granulation tissue or mature fibrous in rare fields in the section examined. Score 0 was given when no granulation tissue or mature fibrous tissue was found in the section.

Epidermal damage: This included erosion to ulceration and/or crusting of the epidermis with inflammatory cells infiltrating the epidermis and adjacent subcutis. Score 5 was never given to any section in this study but was reserved for cases with ulceration of the epidermis and inflammatory cell infiltration involving several fields. Score 4 was given when there was ulceration of the epidermis and inflammatory cell infiltration involving one or two fields and extended into the dermis. Score 3 was given when there was deep erosion of the epidermis and/or inflammatory cell infiltration but restricted to the epidermis. Score 2 was given when there was erosion of the epidermis with or without inflammatory cell infiltration. Score 1 superficial erosion affecting only a few cells in the epidermis. Score 0 was given when no epidermal changes were found.

Factors evaluated: Electric pulse cycles (0-8 pulse cycles); Needle array diameter (0.5, 1.0, and 1.35 cm); Voltage: 0.5 cm and 560V, 0.5 cm and 672 V, 1 cm and 1130 V, 1.35 cm and 1500V).

Results

The morphologic changes found in this study included muscle necrosis, muscle inflammation, muscle hemorrhage, muscle fibrosis, epidermal damage, epidermal inflammation and subcutaneous inflammation.

Muscle Necrosis: Skeletal muscle directly under the skin at the sites specified was the tissue taking the bulk of the action of the treatment applied. Coagulation necrosis of muscle fibers was severe in sections from pigs sacrificed on days 1 and 5, but subsided in pigs sacrificed on day 10 and practically disappeared by day 40. The effect of time after treatment on this change was statistically significant (P=0.0003).

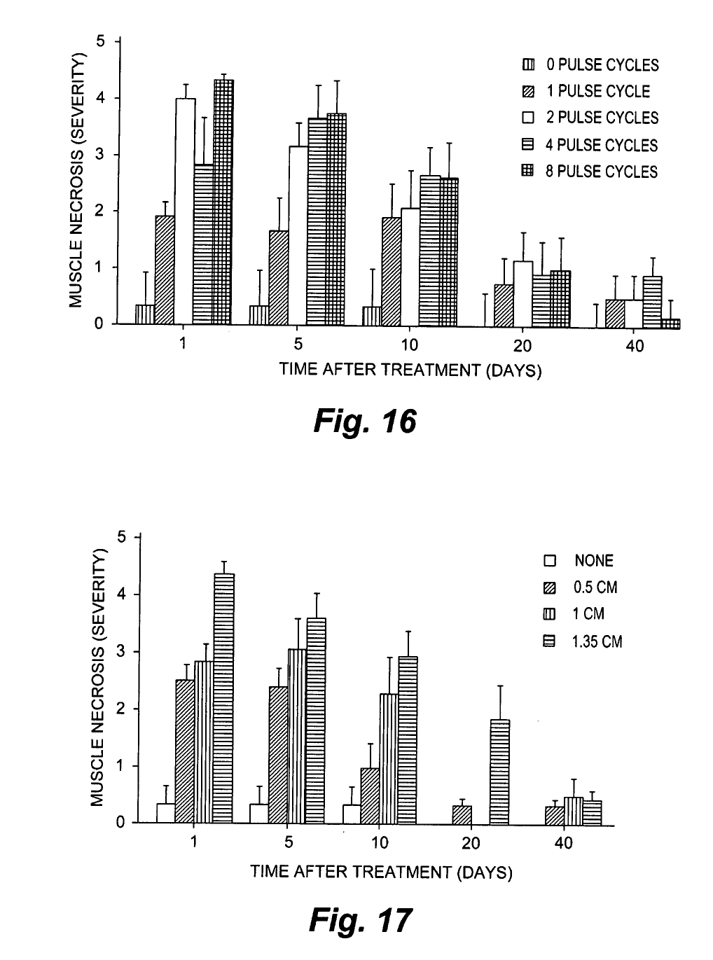

Electric Pulse Cycles: The effect of the number of electric pulse cycles upon the severity of muscle necrosis is complex. While the severity of histological changes increased with the number of pulse cycles applied (P<0.0001), the effect varied depending on the day in which samples were taken (FIG. 16). The statistical interaction between pulse cycle and time after treatment was also statistically significant (P<0.0001).

The interaction between time after treatment and number of pulse cycles is also statistically significant (P<0.03). This means that the magnitude of the difference in severity scores between sections treated with various numbers of pulse cycles vary with time after treatment. To better study the impact of increased numbers of pulse cycles on severity of necrosis, a statistical analysis was applied to data from days 1 and 5. The difference in severity of muscle necrosis between these tow days was not significant (P>0.6), while the severity increased with the number of pulse cycles applied (P<0.0001). Comparing muscle necrosis in different pulse groups by Student's t test, it was found that there was no difference in the necrosis in sections receiving 2, 4, and 8 pulse cycles. But severity scores were higher in these (receiving 2, 4, and 8 pulse cycles) than in sections receiving 1 pulse cycle (P<0.05) and these in turn had more severe histological changes than sections receiving no electrical treatment (P<0.05).

Regression analysis using the number of pulse cycles as a continuous variable in these two days further supported the interpretation that the severity of histological change increased with the umber of pulse cycles used (P<0.001), even if the effect seemed to plateau after 4 pulse cycles.

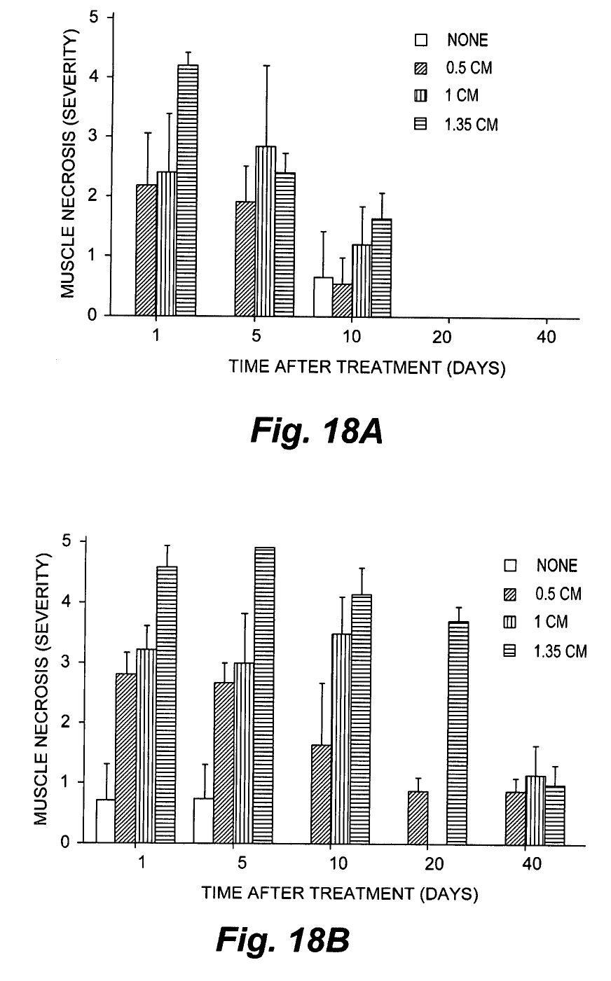

Needle Array Diameter: The effect of the diameter of the needle array used upon the severity of muscle histological change is similar but independent of the effect of the number of pulse cycles applied (interaction between needle array and electric pulse cycles was not significant (P>0.06). While the severity of histological change seemed to increase with the diameter of the needle array used (FIG. 17), that effect was obscured by the interaction between this factor and the type of treatment received. This is because the changes were most severe in sections treated with needle arrays of 1.35 cm diameter than in any other needle array/treatment combination (FIG. 18). Further, the effect of the needle array was also obscured by an interaction of this effect with the effect of time after treatment (P<0.07).

To simplify the study of the effect of needle array on muscle necrosis, days 10, 20, and 40 were excluded from the following analysis. In this analysis, it was shown that the effect of the diameter of the needle array used was highly significant (P<0.001) but there was no difference in severity scores between the days 1 and 5. By Student's t test it was found that the severity scores were highest in sections treated with the 1.35 cm needle arrays and that those scores were significantly different from the scores of sections treated with the 1 and 0.5 cm needle arrays (P<0.05). There was no difference in the severity scores of sections treated with 0.5 cm and 1 cm needle arrays (P>0.05). By comparison, scores in sections not treated with electroporation were negligible (P>0.05).

Regression analysis using the needle array diameter (independent variable) as a continuous variable on days 1 and 5 further supported the interpretation that the severity of changes (dependent variable) increased with the needle array diameter (P<0.001).

Voltage: Generally, all sections treated with a specific needle array received a corresponding voltage. However, sections treated with the 0.5 cm needle array received either 560V or 672 V. A multifactorial analysis of variance showed that muscle necrosis was statistically more severe in sections treated with 672 V than with 560 V (P<0.05) and that this effect was independent of the treatment and the time after treatment.

Muscle Inflammation: In association with the muscle necrosis, an infiltrate of inflammatory cells was found in the muscle tissue.

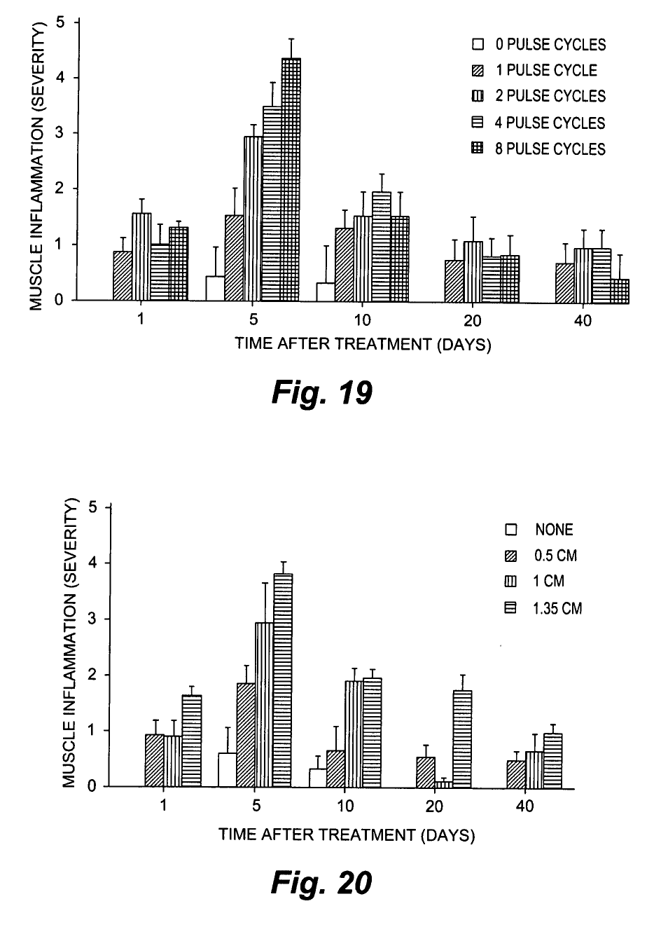

Electric Pulse Cycles: The effect of the number of electric pulse cycles upon the severity of muscle inflammation is shown in FIG. 19. The interaction between the number of electric pulse cycles and the time after treatment was statistically significant (P<0.007), and is illustrated in FIG. 19 by the spike in scores on day 5 after treatment. The effect of the number of pulse cycles upon this change was independent of the treatment and also independent of the diameter of the needle array used (P>0.41).

To better study the impact of increased number of pulse cycles on the severity of inflammation, a statistical analysis was applied to data from day 5 only. The severity of this lesion increased with the number of pulse cycles applied (P<0.0001). By comparison of the muscle necrosis in different pulse groups by Student's t test, it was found that there was no difference in the necrosis in sections receiving 2, 3, and 8 pulses cycles. But severity scores were more severe in these (receiving 2, 3, and 8 pulse cycles) than in section receiving 1 pulse (P<0.05) and these in turn had more severe changes than sections receiving no electrical treatment (P<0.05).

Needle array diameter: The effect of the diameter of the needle array used upon the severity of the muscle inflammation is shown in FIG. 20. The interaction between the diameter of the needle array used and the time after treatment was not statistically significant (P>0.23). The interaction between the diameter of the needle array used and the treatment substance administered was not significant either (P>0.7).

The severity of this lesion increased with the diameter of the needle array (P<0.0001). By Student's t test, it was found that there was no difference in the muscle inflammation in sections treated with needle arrays of diameter 0.5 and 1.0 cm. but severity scores were larger in sections treated with needle arrays of 1.35 cm in diameter (p<0.05) and smaller in sections receiving no electrical treatment (P<0.05).

Muscle Hemorrhage: Hemorrhage was commonly associated with areas of necrosis. The hemorrhage was usually well circumscribed and appeared to reflect the severity of the necrosis. Hemorrhage was only rarely found on day one, presumably because it developed after the necrosis was advanced.

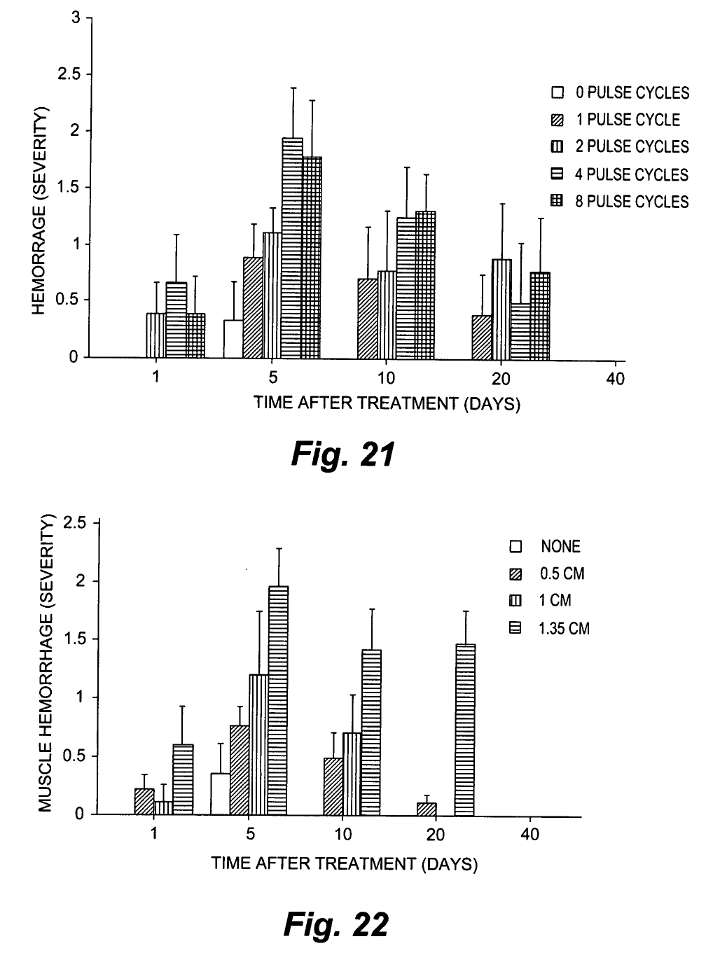

Electric Pulse Cycles: The effect of the number of electric pulse cycles upon the severity of muscle hemorrhage is shown in FIG. 21. The interaction between the number of electric pulse cycles and the time after treatment was not statistically significant (P>0.90). The effect of the number of pulse cycles upon this change was independent of the treatment solution and also independent of the diameter of the needle array used (P>0.92). The severity of hemorrhage increased with the number of pulse cycles applied. By Student's t test it was found that the severity of hemorrhage was greater in sections treated with more than 2 pulse cycles than the severity of hemorrhage in sections treated with 1 pulse or none at all (p<0.05).

Needle Array Diameter: The effect of the diameter of the needle array used upon the severity of the muscle hemorrhage is shown in FIG. 22. The interaction between the diameter of the needle array used and the time after treatment was not statistically significant (P>0.13). The interaction between the diameter of the needle array used and the treatment solution was statistically significant (P<0.002). By Student's t test it was found that hemorrhage was most severe in sections treated with 1.35 cm needle arrays (P<0.05), while others had similar amount of hemorrhage to those undergoing no electrical treatment (P>0.05).

Fibrosis: Immature mesenchymal cells were found in muscle lesions starting at 5 days after treatment, but reduced in severity from moderate to mild subsequently (10 days).

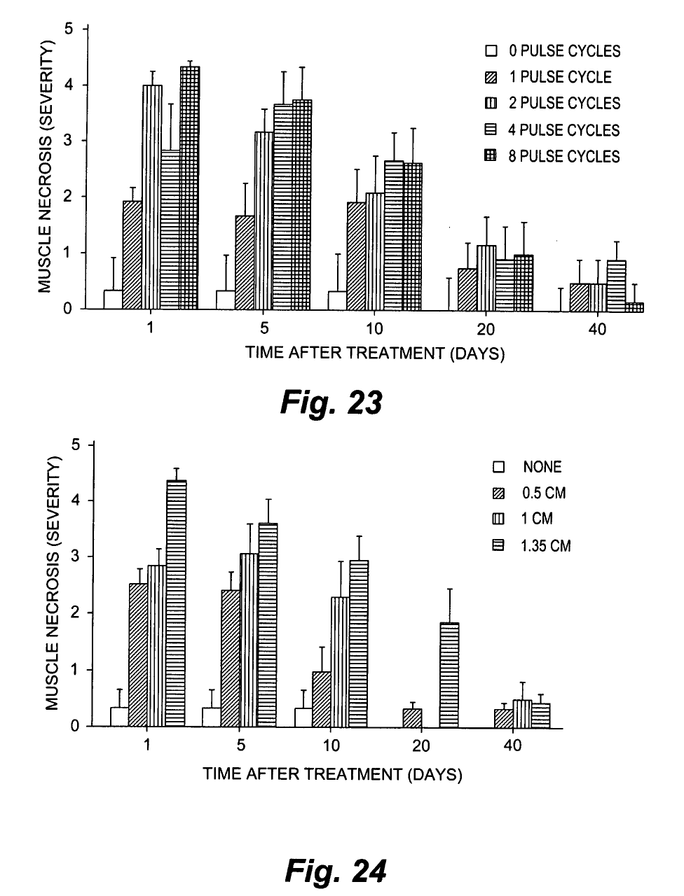

Electric pulse Cycles: The effect of the number of electric pulse cycles upon the severity of muscle fibrosis is shown in FIG. 23. The interaction between the number of electric pulse cycles and the time after treatment was statistically significant (P<0.022). The effect of the number of pulse cycles upon this change was independent of the treatment solution and also independent of the diameter of the needle array used (P>0.86).

The severity of fibrosis increased with the number of pulse cycles applied. By Student's t test it was found that the severity of hemorrhage was greater in sections treated with more than 2 pulse cycles than the severity of hemorrhage in sections treated with 1 pulse or none at all (P<0.05).

Needle Array Diameter: The effect of the diameter of the needle array used upon the severity of the muscle fibrosis is shown in FIG. 24. The interaction between the diameter of the needle array used and the time after treatment was not statistically significant (P>0.08). The interaction between the diameter of the needle array used and the treatment solution was statistically significant (P<0.05). By Student's t test it was found that fibrosis was most severe in sections treated with 1.35 cm needle arrays (P<0.05). Sections treated with needle arrays of 0.5 and 1 cm diameter had similar levels of fibrosis and these were less severe than the fibrosis found in sections treated with 1.35 cm diameter arrays but more severe than sections undergoing no electrical treatment (P>0.05).

Epidermal Damage: This lesions was characterized by epidermal ulceration or erosion.

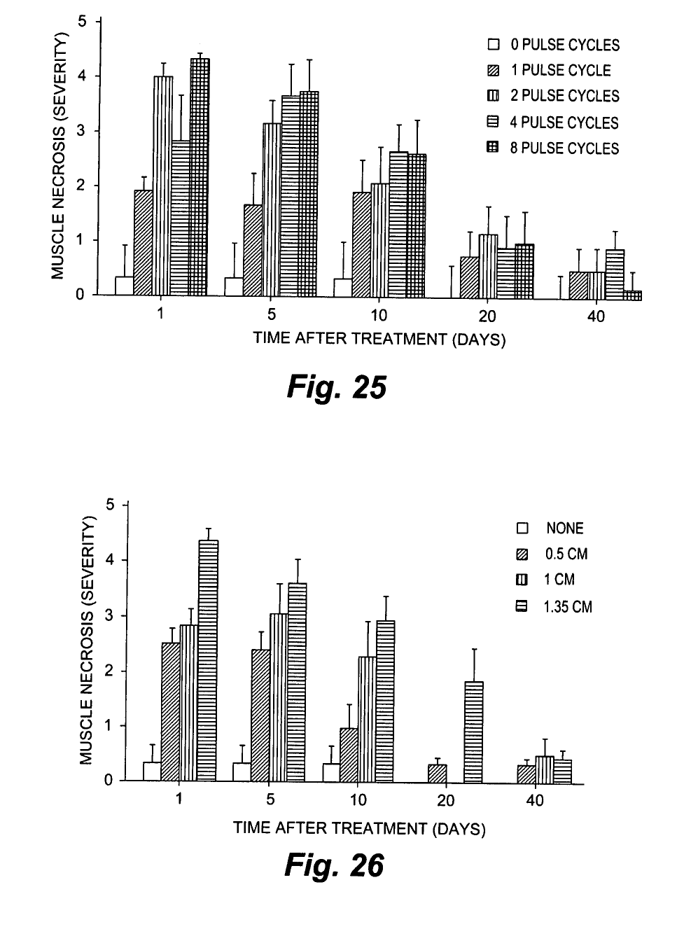

Electric Pulse Cycles: The effect of the number of pulse cycles upon this change was not significant (P>0.24, FIG. 25). Although the changes were significantly less severe in samples receiving no electric treatment than in electrically treated samples (P<0.02), there was no effect related to the various numbers of pulse cycles given (P>0.24).

Needle Array: The effect of the diameter of the needle array used upon this change was significant (P<0.01), FIG. 26). This effect was due to the minimal severity of the epidermal damage in sections receiving no electrical treatment (P<0.05). The epidermal damage found among sections treated with needle array of different diameters was not statistically different (P>0.05).

Epidermal Inflammation: This lesion was characterized by a mild sub-epidermal inflammatory infiltrate directly below the erosion or ulceration of the epidermis described above.

Histological changes were not significantly affected by the number of pulse cycles used (P>0.5) or by the needle array used) P<0.5). The changes were more severe in treatment solution containing bleomycine vs. saline (P<0.05). The changes also varied significantly with the time after treatment, being most significant on the first day after treatment (P<0.0001).

Subcutaneous inflammation: This histological change was characterized by inflammatory cell infiltrate and fibrosis. The change is associated with necrosis or subcutaneous musculature, or with inflammation directly connected to ulceration of the epidermis.

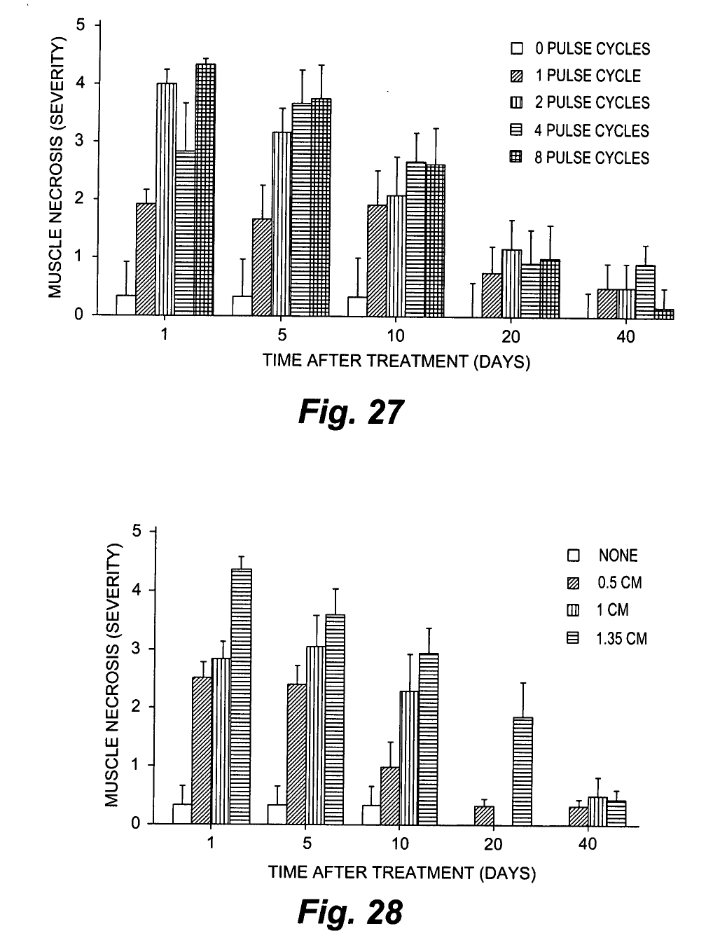

The histologic change was significantly affected by the number of pulse cycles used (P<0.0004), FIG. 27) and by the needle array used (P<0.0001, FIG. 28). Subcutaneous inflammation was more severe in bleomycin treated sections vs saline but only after day 10 (P<0.05). Changes also varied significantly with the time after treatment, being most significant on days 10 and 20 after treatment (P<0.0001).

Discussion

Histologic changes in the form of lesions associated with electroporation and treatment, such as bleomycin, in the skin and subcutis of pigs included severe muscle necrosis, inflammation and fibrosis, mild hemorrhage, mild subcutaneous inflammation and mild to minimal epidermal damage. Severe, but circumscribed lesions were found on days 1 to 10 after treatment, but subsided by day 20 and 40.

Statistical evaluation of the complex interactions of factors studied in this experiment revealed that the effect of the diameter of the needle array and the corresponding voltage used, the number of electric pulse cycles applied and the treatment solution itself, has significant and independent effect upon the lesions found in the subcutaneous muscles. This effect was transitory and lesions were reversed by the 40^{th} day after treatment.

In general the muscle lesions were more severe in sections receiving 2, 4, and 8 pulse cycles than in sections receiving 1 pulse or no electrical treatment. Further, lesions were most severe in sections treated with the 1.35 cm needle array, followed by sections treated with 0.5 and 1 cm needle array, and least severe in sections receiving no electrical treatment. In sections treated with 0.5 cm needle arrays, those receiving 672 V had more severe necrosis than those receiving 560 V. The lack of statistical interaction between number of pulse cycles, needle array diameter, voltage and the treatment solution suggests that all these factors act independently to cause the lesions found and that they may have additive effects.

The epithelial damage and inflammation were mild and likely due to the acute penetration of the needle array through the skin. Interestingly, while the number of electric pulse cycles had no significant effect upon the severity of this lesion, the effect of the diameter of the needle array was significant, being more severe in sections treated with the needle arrays of larger diameters. A direct and temporary toxic effect of bleomycin upon the epidermis is possible, as indicated by the finding. The transitory nature of the lesion indicates that it has low biological impact, other than for the immune stimulation provided thereby.

The subcutaneous inflammation is only a mild change, compared to the lesions found in the subcutaneous muscle, and it is affected by the same factors, namely number of pulse cycles, needle array diameter and blemomycin treatment. Contrary to other findings, this lesion was most significant on days 10 and 20 after treatment. The reason for this is obscure, but because of the relatively mild severity of the lesion, its biological impact is questionable as well.

### EXPERIMENT 2

Another study was carried out in pigs using lower voltages than those above in Experiment 1. Here, plasmid nucleic acid encoding two different antigens were used, namely bovine herpes virus 1 (BHV-1) glycoprotein D gene (gD), a membrane protein and highly immunogenic antigen, and a plasmid expressing hepatitis B surface antigen (HBsAg) which assembles into a 22 nm particle. This allowed a comparison of immune responses to membrane bound and particulate antigens in a single animal. The study also provides data showing that at the lower voltage ranges of between 100 and 200 Volts, there are statistically significant differences in the amount of stimulation of the target immune system. Table 3 lists the different test groups and voltages and pulses applied.

TABLE 3

| | | | # of |
|---|---|---|---|
| Group | Electroporation conditions | Vaccine* | animals |
| 1 | No EP | 100 ug pgD plus 500 ug | 6 |
| | | pHBsAg | |
| 2 | 200 V/20 ms/6 pulses 1 hr | 100 ug pgD plus 500 ug | 6 |
| | before DNA administration | pHBsAg | |
| 3 | 100 V/60 ms/2 pulses | 100 ug pgD plus 500 ug | 6 |
| | | pHBsAg | |
| 4 | 200 V/20 ms/6 pulses | 100 ug pgD plus 500 ug | 6 |
| | | pHBsAg | |
| 5 | 200 V/60 ms/2 pulses | 100 ug pgD plus 500 ug | 6 |
| | | pHBsAg | |
| 6 | No treatment | No treatment | 2 |

*Plasmids were mixed together in 500 ul PBS and administered in one intramuscular injection on days 0 and 28 on opposite sides.

At two-week time points, blood was collected and serum was obtained following centrifugation. Anti-hepatitis B surface antibodies were measured and quantification in milli-international units/ml was performed in parallel. Titers of anti-BHV-1 neutralizing antibody in sera were determined and expressed as the highest dilution of serum that caused a 50% reduction of the number of viral plaques compared to the untreated virus control.

For measurement of cellular responses, porcine blood was collected and peripheral blood mononuclear cells (PBMCs) were isolated by techniques well established in the biomedical arts. Proliferation of gradient-purified cells was measured. For histological examination, muscle samples were obtained from all injection sites using an 8 mm punch, immediately following euthanasia of the test pigs. From pigs immunized with gD and HBsAg DNA, the injection sites of both the primary immunization and the contra-lateral secondary immunization were sampled at 6 and 2 weeks respectively.

Results

Prior to any DNA immunization experiments, gene expression and inflammatory cell infiltration were assessed in quadriceps muscle under the electroporation conditions described in Table 4. Using the luciferase reporter gene, gene expression was determined for each treatment. Pretreatment with electroporation (Group 2) did not significantly change gene expression compared to plasmid administered without electroporation (Group 1). In contrast, different electroporation parameters administered immediately following plasmid administration all increased gene expression similarly in all groups (Groups 3-5) given electroporation.

TABLE 4

| | | | Severity of |
|---|---|---|---|
| | | | histological |
| | Electroporation | Luciferase gene | inflammatory |
| Group | conditions | expression | reaction |
| 1 | No EP | Low expression | Mild (7%) |
| 2 | 200 V/20 ms/6 pulses | Low expression | Severe (33%) |
| | 1 hr before DNA ad- | | |
| | ministration | | |
| 3 | 100 V/60 ms/2 pulses | High expression | Moderate- |
| | | | Severe (24%) |
| 4 | 200 V/20 ms/6 pulses | High expression | Severe (33%) |
| 5 | 200 V/60 ms/2 pulses | High expression | Severe (29%) |
| 6 | Naïve | None | Normal (3%) |

As indicated in Table 4, groups 3 and 5 show that tissue damage after a 100 V treatment is statistically lower than after the use of 200 Volts for the same pulsing parameters.

Histological examination was carried out for each treatment on tissue from the injection sites sampled 48 hr following administration of luciferase encoding plasmid. Plasmid administered without any electroporation (Group 1) caused a mild inflammatory response, assessed by the amount of blue (nuclear) staining, and consisted primarily of macrophages and neutrophils. Electroporation conditions of 200 V/20 ms/6 pulses (Groups 2 and 4) and 200 V/60 ms/2 pulses (Group 5) caused muscle necrosis in addition to severe inflammation (marked influx of macrophages and neutrophils), whereas electroporation conditions of 100 V/20 ms/2 pulses (Group 3) resulted in muscle necrosis with moderate to severe infiltration of macrophages and neutrophils. In all groups treated with electroporation (Groups 2-5), scattered muscle fibers showed degeneration characterized by mildly increased eosinophilia and reduction in diameter.

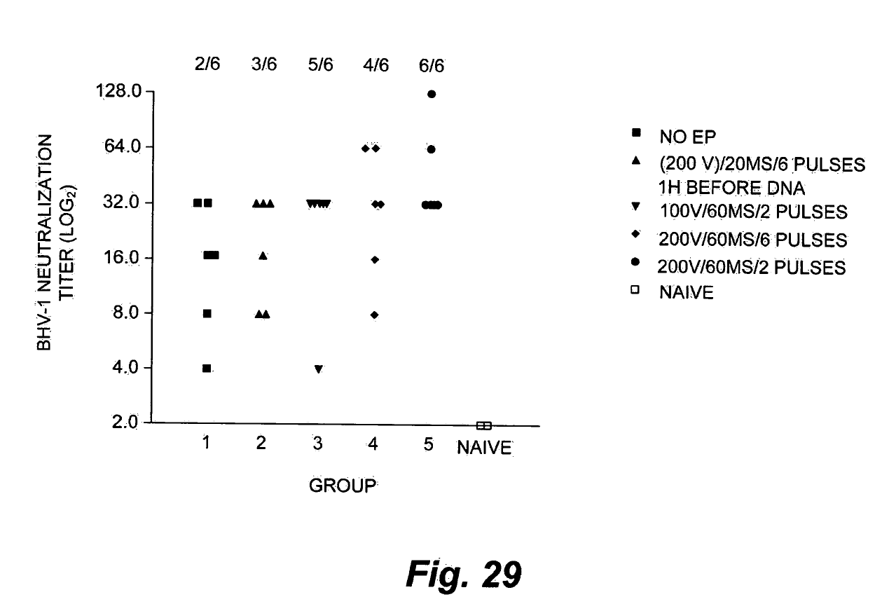

With respect to immune responses, Glycoprotein D-specific antibody responses were determined by BHV-1 neutralization assay. Immunization with plasmid without electroporation (Group 1), conditions that give low gene expression and low cellular infiltration, elicited the lowest number, of animals, only 2/6, achieving a neutralization titer of greater or equal to 32. Animals treated with electroporation one hour prior to plasmid administration (Group 2), showed low gene expression with high cellular infiltration, with similar BHV-1 neutralization antibody responses to Group 2 as shown in FIG. 29. Animals treated with electroporation immediately following immunization (Group 3-5, conditions which gave high gene expression and high cellular infiltration resulted in more animals achieving a neutralization titer of greater or equal to 32 compared to those treated with conventional plasmid immunization.

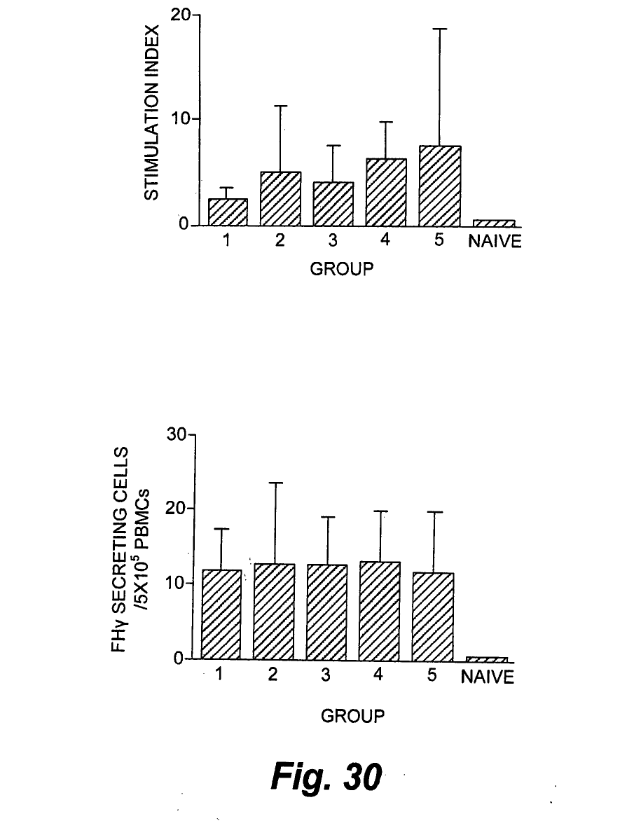

Although gD-specific proliferation responses were not significantly different between the experimental groups as shown in FIG. 30, there was a trend that the lowest stimulation indexes were in animals immunized with no accompanying electroporation (Group 1) whereas groups that received electroporation had higher stimulation indexes (Groups 2-5).

To determine if Th1-like responses were obtained, lymphocytes from immunized pigs were assessed for production of IFN-gamma. DNA immunization with a gD-encoding plasmid stimulated gD-specific interferon gamma secreting cells suggesting a Th1 response, supporting previous reports that DNA vaccines polarize the response towards a Th1-like or balanced response. However, all immunization conditions elicited similar numbers of gD-specific IFN gamma secreting cells.

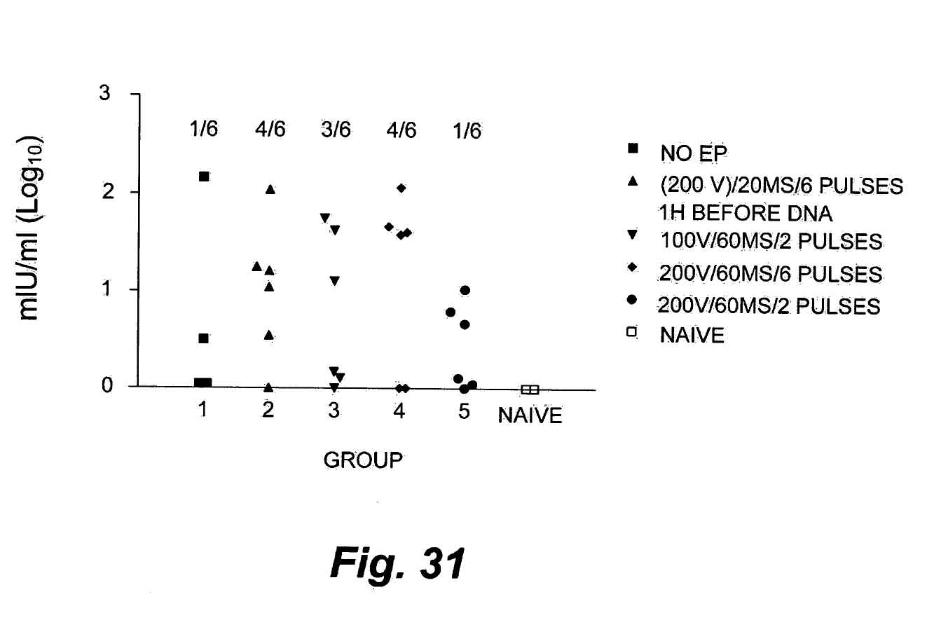

Immune responses to HBsAg were determined using a clinical ELISA test as shown in FIG. 31. Animals immunized without electroporation (Group 1) had the weakest immune responses, with only two animals responding to the immunization and only ⅙ animals responding with a titer considered to be protective (>10 mIU/ml). In groups that received electroporation at the time of DNA immunization (Groups 3-5), more animals responded and Group 4, which received the strongest electroporation treatment, had the most animals considered protected (4/6). In animals that received electroporation 1 hr prior to DNA immunization (Group 2), the immune responses were similar to animals that received an identical electroporation treatment at the time of immunization; with 4/6 animals considered protected despite the low level of antigen expression.

Further, muscle biopsies from animals 2 weeks following the second immunization carried out at day 28 in conjunction with electroporation (Groups 2-5) were examined and showed a greater degree of cellular infiltration than those from animals that received no electroporation (Group 1), (data not provided). In animals treated with electroporation at the time of plasmid administration (Groups 3-5), the cellular infiltration at 2 weeks following the second immunization consisted primarily of aggregates of lymphoblasts surrounding small vessels within the muscle, whereas in animals treated with electroporation prior to plasmid administration, the mild cellular infiltration consisted predominantly of macrophages and neutrophils.

These data provide evidence that electroporation provides for not only enhanced gene expression but also an enhancement of immune responses that can be predetermined and “controlled” for an intended outcome. For example, the inflammatory cell infiltration was demonstrated to be an important component for enhancing immune responses to DNA vaccines since prior treatment with electroporation enhanced immune responses to the HBsAg DNA vaccine but did not increase gene expression. However, the increase in gene expression caused by electroporation is absolutely critical for inducing protective immune responses as demonstrated using the gD DNA vaccine. That the level of antigen produced is critical in the induction of immune responses to DNA vaccines was illustrated previously. Generation of antibody titers that would be considered protective in humans from hepatitis B could be achieved in 100% of animals, under electoporation conditions of 200 V/20 ms/6 pulses and using two administration sites of 500 ug pHBsAg for the primary and secondary immunization. In the current study, with only one administration site of 500 ug pHBsAg for the primary and secondary immunization, the number of animals with titers considered protective was reduced to 66%. Thus, the mechanism by which electroporation enhances immune responses to DNA vaccines is a combination of increased gene expression and increased inflammation with cellular infiltration.

Although the foregoing has been described in detail by way of illustration and example, it will be apparent to one of ordinary skill in the electroporation arts in light of the disclosure that other variations can be envisaged with respect to the above invention embodiments without leaving the scope and spirit of the claims.

We claim:

1. An electroporation device comprising:
a handle in electrical communication with an electric pulse generating source; and
in electrical communication with said handle a head component comprising elements selected from the group consisting of a plurality of electrodes, an injection port, and an electrode safety shield.

2. The device of claim 1 wherein said handle further comprises a pistol grip for aiding use of the device and a trigger mechanism for activating said electrodes.

3. The device of claim 1 wherein said handle further comprises a receptacle in an upper portion thereof for accommodating a syringe and/or hypodermic injection needle.

4. The device of claim 1 wherein said head has a central support substrate having a handle end, a therapeutic end and a central body, said handle end further comprising a connector for connecting to said handle, said therapeutic end comprising a plurality of electrodes and said injection port, said central body having slidably connected there over said safety shield.

5. The device of claim 4 wherein said electrodes are mounted on said support substrate and are selected from the group consisting of a plurality of at least 4 tissue penetrating needle electrodes, non-tissue penetrating meander electrodes, non-tissue penetrating needleless injector electrodes, and an array of a plurality of semi-tissue penetrating microneedle electrodes.

6. The device of claim 5 wherein said electrodes comprise needle electrodes.

7. The device of claim 6 wherein said needle electrodes are patterned on said support in a geometric array.

8. The device of claim 7 wherein said geometric array is selected from the group consisting of a square, a rectangle, a hexagon, and an octagon.

9. The device of claim 8 wherein said geometric array has electrodes of opposing polarity that are between 0.2 and 2.0 cm distant from one another.

10. The device of claim 9 wherein said electrodes extend from a support structure of between 0.4 and 5.0 cm in length.

11. The device of claim 10 wherein said electrodes are between 0.25 and 1.5 mm thick.

12. The device of claim 11 wherein said electrodes comprise a gold exterior surface of between 0.5 and 20 micrometers thick.

13. The device of claim 4 wherein said central support substrate has a bore therethrough from said injection port through said central body and exiting from said body at or near said handle side of said central support substrate.

14. The device of claim 13 wherein said bore exits from said handle side of said central support and extends through said central support substrate in a direction parallel with said electrodes and of a sufficient diameter to allow the passage therethrough of a canula such that when said canula is placed there through, the end of the canula will protrude from said injection port in parallel relation to needle electrodes if present or perpendicular to meander electrodes if present.

15. The device of claim 13 wherein said bore is connected on or near said handle side to an enclosed channel capable of transporting a fluid medium, said channel having first and second ends, said first end connected in fluid communication with said bore and said second end connected in fluid communication with a connector for a hypodermic syringe.

16. The device of claim 4 wherein there is an injection canula connected to said injection port.

17. The device of claim 4 wherein there is a seal ring placed around said injection port.

18. The device of claim 17 wherein said seal ring comprises any combination of a rubber, plastic, strip of adhesive, and resilient material.

19. The device of claim 11 further comprising an electrode guide, said guide comprising a plate connected to said safety shield, said plate having a plurality of bores therein for passage of said electrodes such that as said electrodes are guided through said bores, said guide will keep said electrodes oriented in a parallel direction with relation to one another as they are directed into biologic tissues.

20. The device of claim 19 further comprising a depth limitation element, said depth limitation element capable of prohibiting said safety shield from sliding on said central core in a direction to expose the electrodes more that a predetermined distance.

21. The device of claim 1 wherein said pulse generating source is capable of producing an electric signal having a wave form selected from the group consisting of an exponentially decaying pulse, a square pulse, a unipolar oscillating pulse train, and a bipolar oscillating pulse train.

22. The device of claim 21 wherein said pulse generating source is operable for generating an electric field having a field strength of between approximately 10 V/cm to 20.0 kV/cm.

23. The device of claim 22 wherein said field strength is between about 50 V/cm and 300 V/cm.

24. The device of claim 1 wherein said handle is a linear handle.

25. The device of claim 3 wherein said handle is a linear handle.

26. The device of claim 2 further comprising a lever connected to said handle, said lever capable of controlling an actuator for applying force to a piston addressed to cause, when said lever is activated, the expulsion of a fluid medium comprising a therapeutic agent from a fluid reservoir, said reservoir comprising a syringe or a vial.

27. The device of claim 26 wherein said piston is a syringe plunger.

28. The device of claim 26 wherein said vial comprises a cylindrical container having first and second ends, said first end comprising a resilient seal capable of puncture by a hypodermic needle, said second end comprising a slideable seal capable of being pushed by force from said second end of said cylinder into said cylinder such that when force is applied to said slidable seal, said fluid medium is expelled from said vile when said resilient seal is punctured.

29. The device of claim 28 wherein said slidable seal of said cylinder is in contact with said piston.

30. The device of claim 26 wherein lever, when fully activated contacts said trigger and activates said trigger.

31. A method of delivering a therapeutic agent to a biologic tissue comprising:
contacting said biologic tissue with an electroporation device according to claim 1;
dispensing a therapeutic agent from a fluid container associated with said device into said tissue, and
activating a plurality of electrodes with an electric field generated by a pulse generator attached to said device; and
removing said device from contacting said tissue.

32. The method according to claim 31 wherein said tissue comprises tissue selected from the group consisting of striated muscle, skeletal muscle, smooth muscle, liver, pancreas, lung, throat, skin, breast, prostate, spleen, vascular, cardiac, and tumor tissue.

33. The method of claim 32 wherein said electrodes comprise any one or more of electrode types selected from the group consisting of needle electrodes, meander electrodes, needleless injector electrodes, and shallow surface-tissue penetrating microelectrodes.

34. The method of claim 33 wherein said needle electrodes comprise an array of a plurality of electrodes situated on a central core support and having a spacing about a central injection port of between 0.2 and 2.0 cm diameter.

35. A method of enhancing an immune response in a mammal comprising:
contacting said mammal with an electroporation device according to claim 1;
dispensing a therapeutic agent from a fluid container associated with said device into said tissue, and
activating a plurality of electrodes with an electric field generated by a pulse generator attached to said device; and
removing said device from contacting said tissue, wherein said therapeutic agent further comprises either an antigen, or a nucleic acid.

36. The method of claim 35 wherein said antigen is a polypeptide.

37. The method of claim 35 wherein said nucleic acid encoding said antigen is capable of expression upon delivery via the electroporation into biologic cells of said mammal.

38. A method of enhancing an immune response in a mammal comprising:
contacting said mammal with an electroporation device according to claim 1;
dispensing a therapeutic agent from a fluid container associated with said device into said tissue, and
activating a plurality of electrodes with an electric field generated by a pulse generator attached to said device; and
removing said device from contacting said tissue, wherein said therapeutic agent further comprises a nucleic acid encoding a cytokine, said cytokine capable of stimulating either an inflammatory response or a regulatory response in said mammal.

39. The method of claim 38 wherein said cytokine is selected from the group consisting of IL-2, IFN-Gamma, IL-12.

40. A method of predetermining a histological outcome in a mammal following the electroporation of tissues of said mammal comprising:
Contacting said mammal tissues with a therapeutic substance;
Contacting said tissues with an electroporation device of claim 1;
Administering to said mammal via said electroporation device an electronic impulse of a predetermined Voltage, field strength, pulse length and pulse number sufficient to elicit a predetermined amount of histologic change in said mammal tissue.

41. The method of claim 40 wherein said product of said therapeutic substance is selected from the group consisting of a polypeptide and a nucleic acid.

42. The method of claim 40 wherein said nucleic acid encodes a cytokine or a chemokine.

43. The method of claim 40 wherein said nucleic acid encodes an antigen comprising a peptide or polypeptide.

44. The method of claim 40 wherein said nucleic acid encodes an anti-sense nucleic acid or a silencing (si)RNA.

45. A method of predetermining a histological outcome in a mammal upon the electroporation-assisted administration of a therapeutic substance to said mammal comprising:
Correlating between one another a histological outcome and electroporation parameters selected from the group consisting of voltage, linear dimension between oppositely charged electrodes of an electrode array, number of electric pulses, time length of electric pulse, and time between pulses;
Selecting a set of said electroporation parameters for a given treatment regimen; and
Applying said set of parameters by electroporating a patient tissue;
Wherein said application of selected electroporation parameters provides said histological outcome.

46. The method of claim 45 wherein the voltage is between 10 and 2000 volts.

47. The method of claim 45 wherein the linear dimension between oppositely charged electrodes is a dimension between 0.2 cm and 2.0 cm.

48. The method of claim 45 wherein the number of electric pulses is between 1 and 6.

49. The method of claim 45 wherein the time length of electric pulses is between 10 milli seconds and 100 micro seconds.

50. The method of claim 45 wherein the time between said electric pulses is between 0.1 second and 2 seconds.

51. The method of claim 45 wherein said histological outcome comprises activation of a patient immune system comprising stimulating T cells to release cytokines and/or chemokines, stimulating antibody production to an antigen.

52. An electroporation device comprising:
a handle in electrical communication with an electric pulse generating source; and
in electrical communication with said handle a head component comprising elements selected from the group consisting of a plurality of electrodes, an injection port containing an injection needle, and an electrode safety shield wherein said shield further comprises a directional fitting for orienting a substantially planar electrode and injection needle guide.

53. The device of claim 52 wherein said planar electrode and injection needle guide maintains said electrode and said injection needle at a 90 degree angle with relation to a tissue when said guide is used with the device.

54. The device of claim 52 wherein said planar electrode and injection needle guide provide for limiting a depth to which said electrodes and/or needle may be inserted into a patient tissue.

55. A needle electrode and injection needle direction and depth guide comprising:
A rigid planar substrate having first and second surfaces and a plurality of through holes in spaced geometric relation to one another bored therethrough, said first surface comprising a smooth planar surface, said second surface comprising extended portions thereof, said extensions forming an extension on the same side as said second surface of said substrate in a direction 90 degrees to said plane to a predetermined distance out of said plane for extending the length therethrough of said bores to a top end, such predetermined distance measured from the first surface.

56. The guide of claim 55 wherein said rigid planar substrate comprises a plastic material.

57. The guide of claim 55 wherein said first surface has applied thereto on selected areas thereof a semi-permanent adhesive said adhesive having a semi-waterproof quality.

58. The guide of claim 55 wherein said adhesive is compatible with use on skin.

59. The guide of claim 55 wherein each of said extended portions comprises a bore therethrough.

60. The guide of claim 59 wherein said bores each comprise a diameter of between 0.5 and 2.5 millimeters.

61. The guide of claim 55 wherein said bores are all aligned in a parallel direction in relation to one another and said bores are collectively aligned 90 degrees to the direction of said planar substrate.

62. The guide of claim 55 wherein said bores are arranged in a geometric pattern.

63. The guide of claim 62 wherein said bores include electrode bores which said electrode bores form a geometric pattern selected from the group consisting of a square, rectangle, triangle, pentagon, hexagon, octagon.

64. The guide of claim 62 wherein said geometric pattern includes electrode bores positioned at the corners of each of said patterns such that said electrode bores can comprise 3, 4, 5, 6, or 8 such electrode bores.

65. The guide of claim 64 wherein the bores are spaced about between 0.2 and 2.0 cm to the next nearest bore on said substrate.

66. The guide of claim 62 wherein one of said bores comprises a syringe injection needle bore, said injection needle bore located centrally with respect to an array of electrode bores.

67. The guide of claim 55 further comprising visual orientation markings selected from the group consisting of color, shape, lines, and dots, place on the surface of said guide.

68. The guide of claim 55 further comprising an electrically conducting material contacting any of said top end of said extensions.
